(19) United States
(12) Patent Application Publication
(10) Pub. No.: US 2025/0057772 A1
(43) Pub. Date: Feb. 20, 2025

(54) ORAL DOSAGE FORM WITH IONICALLY CHARGEABLE HYDROGEL FOR DELIVERY OF ACTIVE AGENT

(71) Applicant: Entrega Inc., Boston, MA (US)

(72) Inventors: S. Randy HOLMES-FARLEY, Arlington, MA (US); John JANTZ, Arlington, MA (US); Jacob Matthew GRAHAM, Newport, RI (US); Daniel BONNER, Sharon, MA (US)

(21) Appl. No.: 18/928,469

(22) Filed: Oct. 28, 2024

Related U.S. Application Data
(63) Continuation of application No. PCT/US23/20565, filed on May 1, 2023.
(60) Provisional application No. 63/337,246, filed on May 2, 2022.
(60) Provisional application No. 63/394,107, filed on Aug. 1, 2022.

(51) Int. Cl. A61K 9/48

(52) U.S. Cl. CPC A61K 9/4808; A61K 9/4866; A61K 9/4891

(57) ABSTRACT

The present disclosure provides, inter alia, a pharmaceutically acceptable oral dosage form comprising an ionically chargeable hydrogel and a protective coating for delivery of an ionically chargeable active agent to an intestinal site. Also provided are methods for treatment using the pharmaceutically acceptable oral dosage form disclosed herein.

### CROSS REFERENCE TO RELATED APPLICATIONS

The present application is a continuation of PCT/US2023/020565, filed on May 1, 2023, which claims benefit of U.S. Provisional Patent Application Ser. No. 63/337,246, filed on May 2, 2022, and U.S. Provisional Patent Application Ser. No. 63/394,107, filed on Aug. 1, 2022. The contents of above applications are incorporated by reference herein in their entireties.

### FIELD OF THE INVENTION

Aspects of the present invention relate to pharmaceutically acceptable oral dosage forms for delivery of an active agent to an intestinal site.

### BACKGROUND

Oral dosing of active agents is attractive for many reasons, including ease of administration and high patient compliance. However, for some active agents, such as poorly absorbed, sensitive (i.e., pH sensitive, enzyme-sensitive, and the like), and/or high molecular weight active agents, oral dosing may be less effective or ineffective for achieving sufficient blood concentration of the active agent as compared to alternative dosing strategies. For example, active agents such as proteins and other macromolecules may be enzymatically degraded in the gastrointestinal tract and/or may have limited transport across the intestinal epithelium.

One potential strategy for circumventing the hostile environment of the gastrointestinal tract is to alter the environment through the use of protease inhibitors and/or derivatization of agents with polyethylene glycol to prevent enzymatic degradation. Another potential strategy is to increase the permeability of the tissue in the gastrointestinal tract such that absorption of an agent increases. An agent may be formulated with an excipient that can, for example, open the tight junctions of the intestine to allow an agent to pass through the intestinal epithelium. A further approach to improving delivery of an agent in the gastrointestinal tract is to apply an enteric coating to the agent such that the agent is not exposed to the harsh pH conditions of the stomach, and is instead released in the small intestine, where absorption occurs more readily.

Another technique for drug delivery is the use of hydrogels as a part of a drug delivery system. However, a need remains for drug delivery systems that are capable of providing improved delivery of an agent to the gastrointestinal tract, such as in a form that allows the active agent to be readily absorbed by the intestinal tissue, without excessive degradation thereof. A need also remains for drug delivery systems that are capable of providing improved active agent delivery to the intestinal tract.

### SUMMARY

According to one embodiment herein, a pharmaceutically acceptable oral dosage form for delivery of an active agent to an intestinal site is provided. The dosage form comprises a delivery device, and a protective coating covering the delivery device. The delivery device comprises: a plurality of hydrogel particulates comprising ionically chargeable hydrogel material, the ionically chargeable hydrogel material comprising a crosslinked polymer material having a Swelling Ratio of at least 5; and an ionically chargeable active agent, the ionically chargeable active agent having a net ionic charge with a sign that is the same as a sign of a net ionic charge of the ionically chargeable hydrogel at an intestinal pH in a range of from about 4 to about 8. Methods for treating disorders in a subject using such dosage form are also provided.

### BRIEF DESCRIPTION OF THE DRAWINGS

To facilitate further description of the embodiments of this disclosure, the following drawings are provided to illustrate and not to limit the scope of the disclosure.

FIG. 1 shows the schematic structure of Hydrogel #3.02. The structures of the two constituents, AMPS and Methylenebisacrylamide, are also shown.

FIGS. 2A-2B show the swelling results of hydrogels with varying amounts of crosslinker in both DI water (A) and pH 7 FASSIF (B).

FIGS. 3A-3B show the exclusion effect (A, bar chart; B, individual points) of hydrogels with varying amounts of crosslinker in pH 7 FASSIF. 1 mg/mL GG-427 was used as the agent. Error bars=1 standard deviation. T-test comparing exclusions to standard Hydrogel #3.02. Exclusions are not statistically different (all p-values>0.05).

FIG. 4 is a schematic showing that high negative charge of Hydrogel #3.02 deters entry of negatively charged molecules.

FIG. 5A-5B show the concentrations of GG-353 in blood samples (A) and its bioavailability ratios (B) via oral delivery to dogs with and without Hydrogel (300 mg).

FIG. 6 show the concentrations of Atenolol in blood samples via oral delivery to dogs with and without Hydrogel (300 mg).

FIGS. 7A and 7B show the result of GG-353 (5 mg) oral bioavailability vs hydrogel content. All Formulations contain: Enteric gelcap (1×size 00), GG-353 (5 mg), atenolol (1 mg) and citric acid (0.25 mg), Sodium caprate (200 mg), soybean trypsin inhibitor (50 mg), and Hydrogel #3.02 (0 to 450 mg).

FIG. 8 is a schematic showing exemplary peptide exclusion rationale with an anionic hydrogel.

FIG. 9 is a schematic showing an embodiment of an oral dosage form comprising ionically chargeable hydrogel and ionically chargeable active agent.

FIG. 10 shows the results of oral bioavailability studies with ionically chargeable hydrogel and three different ionically chargeable peptides.

FIG. 11A is a schematic illustration of an embodiment showing a mixture of protease and ionically chargeable hydrogel, and optionally ionically chargeable peptide, and which mixture can be used to detect a peptide level in the bulk fluid outside the hydrogel (in a case where ionically chargeable peptide, such as peptide C, is added to the mixture), and can be used to detect protease activity in the bulk fluid outside the hydrogel (in which case ionically chargeable peptide is not require to be added to the mixture).

FIG. 11B shows the results for detected ionically chargeable peptide levels for a mixture of the peptide in fluid with protease and hydrogel, and a mixture of the peptide in fluid with protease and without hydrogel (left hand upper graph is peptide concentration for combination with Chymotrypsin, right hand upper graph is peptide concentration for combination with Trypsin); and shows the results for detected protease activity in a mixture of protease with hydrogel in a fluid, protease without hydrogel in a fluid, and a mixture of protease with hydrogel having a net ionic charge with a sign that is the same as that of the protease in a fluid (left hand lower graph is Chymotrypsin activity, right hand lower graph is Trypsin activity).

FIG. 12 shows the schematic structure of Hydrogel #12.035, with the circled part highlighting the difference from Hydrogel #3.02. The structures of the three constituents, AMPS, Methylenebisacrylamide, and Styrene sulfonate are also shown.

FIG. 13 shows the relative concentration of the Evans Blue dye when hydrogel #12.035 is added in varying amounts sufficient to absorb specific fractions of the fluid present in a simulated intestinal fluid at pH 7.

FIG. 14 shows the concentrations of Peptide C in blood samples (left) and its bioavailability (right) via oral delivery to dogs with and without Hydrogel #12.035 (300 mg).

FIG. 15 shows an in vitro study comparing Hydrogel #12.035 and Hydrogel #3.02 using Peptide C.

Other aspects, embodiments and features of the inventive subject matter will become apparent from the following detailed description when considered in conjunction with the accompanying drawing. The accompanying figures are schematic and are not intended to be drawn to scale. For purposes of clarity, not every element or component is labeled in every figure, nor is every element or component of each embodiment of the inventive subject matter shown where illustration is not necessary to allow those of ordinary skill in the art to understand the inventive subject matter.

### Definitions

“Agent” as used herein refers to any treatment agent that can be administered to a patient for treatment and/or prevention of a disease and/or condition, including but not limited to a pharmaceutical agent, a drug, a small molecule drug, a drug conjugate, a prodrug, an antibody or an antibody fragment, a nucleic acid, a protein, a peptide, a polysaccharide, a small organic molecule (e.g., with a molecular weight of about 500 Da or less), a metabolically activated agent (e.g., a metabolite), a nutrient, a supplement, and the like, unless specified otherwise.

“Biodegradable” as used herein refers to materials that, when introduced into the body of an individual, patient, or subject, are broken down by cellular machinery, chemical processes (e.g., hydrolysis), or physical processes (e.g., dissolution) into components (sometimes referred to as “degradation products”) that the body can either reuse or dispose of without significant toxic effect. In some instances, the degradation products may also be biocompatible.

“Mucoadhesive” as used herein refers to a composition having the capacity to bind to a mucosal surface.

“Hydrogel” as used herein refers to hydrophilic crosslinked polymeric structures that are capable of absorbing fluids.

“Dried hydrogel” or hydrogel in a “Dried State” as used herein refers to hydrogel material having a water content that is the same as that for hydrogel material that has been dried for at least 18 hours in a convection oven set to 150° F. at standard pressure.

“Swelling Ratio” as used herein is a measure of the mass of fluid taken up by a sample of hydrogel at a point in time following introduction of the fluid to the hydrogel sample, divided by the initial mass of the hydrogel sample. The Swelling Ratio can be expressed as follows: Q (Swelling Ratio)=(Swollen Mass-Initial Mass)/Initial Mass, or otherwise stated as the ratio of the mass of fluid taken up by the hydrogel (A fluid) to the Initial Mass of the hydrogel (i.e. before introduction of the fluid). The method used to determine the Swell Ratio for a mass of hydrogel, such as a hydrogel particulates, is as follows (performed at approximately standard temperature and pressure):
a. Add 0.1 g dried hydrogel (Initial Mass) to 15 mL fluid;
b. Allow to soak for 15 minutes with gently rocking;
c. Drain unabsorbed fluid via 500 μm mesh; and
d. Measure mass of unabsorbed fluid, to obtain the mass of fluid absorbed by the hydrogel (Δ fluid=initial mass of fluid−mass of unabsorbed fluid). The mass of the fluid (initial or unabsorbed) can be obtained by weighing, or multiplying the volume of the fluid by its density. The fluid used to calculate the Swelling ratio can be any suitable fluid, such as deionized water or Fasted State Simulated Intestinal Fluid (“FASSIF”). For example, a typical FASSIF (e.g., 1 L) used herein can be prepared by adding 961 g DI H₂O to 42 g FASSIF buffer concentrate (Biorelevant Media FASBUF available from Biorelevant.com Ltd, Suite 4, Queen Mary Innovation Center, 42 New Road, London E1 2AX UK), adding 2.24 g FASSIF powder (Biorelevant Media FFF02 available from Biorelevant.com Ltd, Suite 4, Queen Mary Innovation Center, 42 New Road, London E1 2AX UK), stirring until dissolved, and adjusting to desired pH (e.g., 6.5) using 1M NaOH or HCl. The resulting FASSIF typically contains: 3 mM Taurocholate, 0.75 mM Soybean Lecithin, 148 mM Sodium, 106 mM Chloride, and 29 mM Phosphate.

“Swell Ratio Percentage” as used herein refers to the percentage of the Maximum Swell Ratio that a Swell Ratio corresponds to as measured at a select time interval. For example, for a Maximum Swell ratio of 100 for a hydrogel sample, a Swell Ratio of 50 as measured at a time interval of 1 minute would correspond to a Swell Ratio Percentage of 50%.

“Swelling Speed” as used herein refers to the speed with which a hydrogel sample reaches a predetermined Swell Ratio Percentage. For example, a hydrogel sample may have a Swelling Speed such that it reaches a Swell Ratio Percentage of at least 30% in 1 minute, a Swell Ratio Percentage of 50% in 2 minutes, and a Swell Ratio Percentage of 100% in 10 minutes.

“Exclusion Amount” as used herein refers to an amount of active agent excluded from a hydrogel material, when the hydrogel material is introduced to a Fasted State Simulated Intestinal Fluid (FASSIF) (the FASSIF medium described above at pH of 6.5). The Exclusion Amount is calculated by:
a. Determining a mass of the hydrogel material needed to absorb 8 mL of FASSIF, using the Swelling Ratio of the hydrogel material;
b. Placing the calculated amount of hydrogel in 10 mL of 1 mg/mL active agent in 15 mL tube under gentle rocking (active solution).
c. Placing the calculated amount of hydrogel in 10 mL of pH 7 FASSIF in 15 mL tube under gentle rocking (background solution).
d. Every 5 minutes for 25 minutes total, extracting 3×10 μL samples and diluting into 190 μL deionized water in 96-well UV plate.
e. Also taking 3×10 μL samples of 1 mg/mL active agent and pH 7 FASSIF stock solutions.
f. After completion, measuring absorbance of each sample at 230 nm and 265 nm (or another absorbance characteristic of the active agent).
g. Subtracting the background from the active absorbance readings at each time point, and comparing to stock solution to calculate the Exclusion Amount.

“Dried hydrogel” or hydrogel in a “Dried State” as used herein refers to hydrogel material having a water content that is the same as that for hydrogel material that has been dried for at least 18 hours in a convection oven set to 150° F. at standard pressure.

“Ionically chargeable” as used herein means that the hydrogel material and/or active agent contain functional groups that are capable of being in charged form when the material/agent is provided in an aqueous medium, such as gastrointestinal fluid, deionized water, or FASSIF.

“Net ionic charge” as used herein in reference to an ionically chargeable material, such as a hydrogel comprising ionically chargeable groups and/or an active agent having ionically chargeable groups, provided in an aqueous medium such as a gastrointestinal fluid, deionized water, or FASSIF, means the sum of all ionic charges of the chargeable groups that adopt a charged state in the aqueous medium. The sign of the net ionic charge of the material may be either positive, negative, or neutral, depending on the whether the material has more negatively charged groups, more positively charged groups, or no charged groups, as well as the magnitude of charge of any charged groups, when provided in the aqueous medium. For example, a material with primarily ionically chargeable groups that adopt a negative charge when provided to the aqueous medium may have a net negative ionic charge (the sign of the net ionic charge is negative), whereas a material with primarily ionically chargeable groups that adopt a positive charge when provided to the aqueous medium may have a net positive ionic charge (the sign of the net ionic charge is positive).

“Individual,” “patient,” or “subject” as used herein are used interchangeably and refer to any animal, including mammals, preferably mice, rats, guinea pigs, and other rodents; rabbits; dogs; cats; swine; cattle; sheep; horses; birds; reptiles; or primates, such as humans.

“Pharmaceutically acceptable carrier” or “pharmaceutically acceptable excipient” as used herein refers to any and all solvents, dispersion media, coatings, isotonic and absorption delaying agents, and the like, that are biocompatible and otherwise suitable for administration to an Individual.

“Pharmaceutical composition” as used herein refers to a composition comprising at least one agent as disclosed herein formulated together with one or more pharmaceutically acceptable carriers and/or excipients.

“Pharmaceutically or pharmacologically acceptable” as used herein refers to molecular entities and compositions that are acceptable for administration to an animal, or a human, as appropriate, for example in not producing an excessive adverse, allergic, or other untoward reaction.

“Treating” as used herein refers to any effect, for example, lessening, reducing or modulating, that results in the improvement of the condition, disease, disorder, and the like.

“Protease Activity Assay,” as used herein refers to an assay that determines the protease activity in a given system, as understood by those of ordinary skill in the art. An example of two such assays are a chymotrypsin or trypsin assay. Protease activity for proteases comprising chymotrypsin or trypsin can be determined, for example by a Protease Activity Assay using either alpha-chymotrypsin, CAS: 9004-07-3 or Trypsin Type 1, CAS: 9002:07-7. According to one embodiment, a chymotrypsin activity assay kit is used, such as that available from Abcam, including coumarin substrate, chymotrypsinogen activator, and a chymotrypsin assay buffer (“cTAB”). According to one embodiment, a trypsin activity assay kit, such as that available from Abcam, including p-NA substrate, and Trypsin assay buffer (“TAB”). Materials for the assay can further comprise Tris solution having a pH of 7.4 with 0.5 mM Ca.

In one embodiment, to prepare the Protease Activity Assay for chymotrypsin, the following steps are performed: mass the hydrogel (such as hydrogel #3.02 described in Example 1, having particle sizes in the range of 500 to 2000 microns) in 75 mg aliquots into a labeled 15 mL tube; label an additional 15 mL tube as the control; create aliquots of the reaction solution (e.g. per Abcam assay kit instructions) by combining 46 microliters of cTAB, 2 microliters of Coumarin substrate, and 2 microliters of chymotrypsinogen activator in each aliquot (e.g. to provide for 4 timepoints, n=3 all samples, 2 tubes=24 aliquots total required, so total solution is 1104 microliters of cTAB, 48 microliters of Coumarin substrate, and 48 microliters of chymotrypsinogen activator); vortex the reaction solution to mix; create the sample background control (SBC) solution (e.g. per Abcam assay kit instructions) by combining, for one aliquot, 48 microliters of cTab and 2 microliters of chymotrypsinogen activator (e.g. to provide for 4 timepoints=4 aliquots required, so total solution is 192 microliters of cTab and 8 microliters of chymotrypsinogen activator); vortex solution to mix; and create 10 ug/mL chymotrypsin solution in tris buffer (e.g., 15 mL total solution for test with 5 mL per tube). The procedure for the chymotrypsin activity assay includes adding 5 mL of chymotrypsin solution in tris buffer to each of the sample tubes containing dry hydrogel, and empty control tubes, beginning the timer and mixing with a spatula to break up any hydrogel as needed. The tubes are then incubated at 150 RPM and 39° C. Sampling from each of the tubes is performed at each time point (5, 15, 30 and 60 minutes) by taking 10 microliters from each tube, and combining into 50 microliters of the reaction solution described above, with n=3 tubes, for a total of 6 samples per time point. The samples are provided in wells of a UV-clear, flat-bottom plate (e.g. 96 well plate), and 50 microliters of the SBC solution above is provided in a single well as a moving background. The fluorescent readings are read at 360/460 nm for the respective sample wells at the time points. For each tube at each time point and incubation, the three readings for a single output are averaged, and the SBC solution reading is subtracted from each of the averages. For each average after the SBC solution reading is subtracted, calculation of the activity relative to the initial control reading is made for each incubation.

In one embodiment, to prepare the Protease Activity Assay for trypsin, the following steps are performed: mass the hydrogel (such as hydrogel #3.02 described in Example 1, having particle sizes in the range of 500 to 2000 microns) in 75 mg aliquots into labeled 15 mL tube; label an additional 15 mL tube as the control; create aliquots of the reaction solution (e.g. per the Abcam assay kit) by combining 48 microliters of TAB and 2 microliters of p-NA substrate in each aliquot (e.g. to provide for 4 time points, n=2 all samples, 3 tubes=26 aliquots total required, so total solution is 1248 microliters of TAB and 52 microliters of p-NA substrate); vortex the reaction solution to mix; and create a 150 microgram per liter trypsin solution in tris (e.g. per Abcam assay kit instructions) with 5 mL per tube for a total of 15 mL. The procedure for the trypsin activity assay includes adding 5 mL of trypsin solution in tris buffer to each of the sample tubes containing dry hydrogel, and empty control tubes, and beginning the timer and mixing with a spatula to break up any hydrogel as needed. The tubes are then incubated at 150 RPM and 39° C. Two 50 microliter reaction solution aliquots are place in a separate well of the plate as a background. Sampling from each of the tubes is performed at each time point (5, 15, 30, 60 minutes). Each of the 50 microliters sample is placed in 450 microliters of TAB and vortexed to mix. Then from each solution 10 microliters is taken from each tube, and combining into 50 microliters of the reaction solution described above. The samples are provided in wells of a UV-clear, flat-bottom plate (e.g. 96-well plate). The fluorescent readings are read at 405 nm for the respective sample wells at the timepoints. For each tube at each timepoint and incubation, the average of 2 readings for a single output are averaged, and the average of the reaction solution background readings is subtracted from all averages. For each average after background subtraction, calculation of the activity relative to the initial control reading after background subtraction is made for each incubation.

“Active Agent Concentration Assay,” as used herein includes any assay suitable to determine active agent concentration (such as peptide concentration) in a fluid sample that is known to those of ordinary skill in the art. For example, a suitable assay to determine a concentration of active agent in a fluid when provided in combination with an ionically chargeable hydrogel may comprise mixing the ionically chargeable hydrogel material and active agent in the fluid (e.g. water), optionally with any other compounds of interest such as protease. The ionically chargeable hydrogel material can be allowed to settle to the bottom of the mixture (e.g. the bottom of a test tube containing the mixture), and the fluid at the top of the mixture above the hydrogel can be sampled. The concentration of the active agent that is present in such a sample can be determined by a standard method such as liquid-chromatography-mass spectrometry (LCMS).

The singular forms “a,” “an,” and “the,” as used herein, include plural referents unless the context clearly dictates otherwise.

The terms “comprising,” “comprises,” “including,” and “includes” should be interpreted as referring to elements, components, or steps in a non-exclusive manner, indicating that the referenced elements, components, or steps may be present, utilized, or combined with other elements, components, or steps that are not expressly referenced.

### DETAILED DESCRIPTION

Aspects of the present disclosure are directed to dosage forms, systems and methods for the oral, trans-intestinal, and/or trans-mucosal delivery of an active agent. In particular, aspects of the present disclosure relate to a pharmaceutically acceptable oral dosage form for delivery of an active agent to an intestinal site comprising a delivery device, and a protective coating covering the delivery device. The delivery device comprises: a plurality of hydrogel particulates comprising ionically chargeable hydrogel material, the ionically chargeable hydrogel material comprising a crosslinked polymer material having a Swelling Ratio of at least 5; and an ionically chargeable active agent, the ionically chargeable active agent having a net ionic charge with a sign that is the same as a sign of a net ionic charge of the ionically chargeable hydrogel at an intestinal pH in a range of from about 4 to about 8. Aspects of the present disclosure also relate to dosage forms comprising ionically chargeable hydrogel in a form with physical properties that allow for excellent active agent delivery characteristics at an intestinal site, such as swelling ratio, swelling speed, compressive strength and radial strength. Further aspects of the present disclosure provide for methods of manufacturing dosage forms with delivery structures containing ionically chargeable hydrogel, as well as methods of administering active agents with the dosage forms.

Without being limited to any theory, it is believed that aspects of the dosage form herein may be capable of providing for enhanced bioavailability of active agents delivered via the dosage form. For example, the dosage form may provide a highly swellable body that is capable of rapidly expanding at an intestinal site, such that at least a portion of the exterior surface of the hydrogel body is pressed into contact with neighboring intestinal tissue at the intestinal site. By contacting the exterior surface of the hydrogel body with the intestinal tissue, the active agent at the exterior surface may be physically contacted with the intestinal tissue and/or placed in close proximity with the intestinal tissue, thereby allowing the intestinal tissue to more readily absorb the active agent to provide enhanced bioavailability of the active agent.

Yet another advantage of the embodiment of the dosage form and/or delivery method described herein may be to reduce the amount of active agent needed for agents which are required to be systemically available (that is, to enter the bloodstream) to be effective. For example, an agent that is only 40% bioavailable in a standard oral dosage form may have higher bioavailability when dosed as described according to embodiments disclosed herein. Higher oral bioavailability has the potential to reduce costs of the active agent, reduce side effects caused by active agent in the GI tract and to reduce the potential for development of side effects due to active agent remaining in the GI tract. Additionally, increasing the oral bioavailability of oral antibiotics has the potential to reduce the development of antibiotic drug resistance due to unabsorbed drug in the small intestine and colon.

Moreover, without being limited to any one theory, it is believed that improved bioavailability of the active agent may be enhanced by the fluid uptake of the hydrogel at the intestinal site, which may increase the effective local concentration of the active agent, providing a greater driving force to transport the active agent across the intestinal wall. Additional potential benefits for bioavailability that may be imparted by hydrogel fluid uptake and/or presentation of the active agent near the mucosal surface, can include the fact that a smaller distance may be required for the active agent to diffuse from the dosage form to the mucosal surface, thus increasing its potential rate of absorption, and also providing for less duration of exposure of the active agent to the harsh and potentially degrading environment of the GI tract.

Furthermore, without being limited to any one theory, it is believed that the ionically chargeable hydrogels of the present invention may increase oral bioavailability of drugs having a net ionic charge with a sign that is the same as a sign of the hydrogels (e.g., an ionically chargeable peptide). For example, when an anionic drug and an anionic hydrogel are released in the small intestine, the hydrogel absorbs moisture, reducing the amount of free fluid present in the local region of the small intestine of the patient. As the hydrogel swells, it may also absorb some drug, but the concentration of drug in the water inside of the hydrogel is lower than it is in the water outside the hydrogel. There are at least two possible mechanisms for the selective uptake of water relative to drug. The first mechanism of drug exclusion from the hydrogel is by ionic interactions, known in the literature as the Donnan Equilibrium or Donnan Exclusion Effect. In short, the tethered negative charges on the hydrogel tend to repel the free negatively charged species (e.g., the anionic drug) from entering the hydrogel. The second mechanism of drug exclusion from the nonporous hydrogel relates to the generally much slower diffusion of larger molecules relative to the faster diffusion of water. Consequently, when the nonporous hydrogel first contacts a fluid containing both water and drug, the diffusion of water into the hydrogel is expected to be faster than the diffusion of large drug molecules. For example, using the known diffusion coefficient of water (˜2.9×10⁻⁹ m²/sec) and the reported diffusion coefficient (0.074×10⁻⁹ m²/sec) of a peptide of similar size, (i.e., 4.5 to 5 kDa), the mean time for water to diffuse 0.5 mm (thus diffusing to the center of a 1 mm particle) is ˜0.7 minutes, while the peptide drug will take 28 minutes to diffuse the same distance. Consequently, the deep interior of the hydrogel particle is expected to be substantially enriched in water relative to drug when it is initially swelling. For at least these two possible reasons, the concentration of drug is higher in the remaining free fluid after hydrogel swelling than it would have been in the absence of the hydrogel in the formulation. Since the uptake of peptide drugs is driven by diffusion and the rate of diffusion is directly related to the concentration by Fick's Law, the increased drug concentration outside of the hydrogel is expected to increase the rate of uptake of said drug. For example, in one embodiment of the present disclosure, a ratio of a fraction of the ionically chargeable active agent taken up into the crosslinked polymer material to a fraction of the water taken up into the crosslinked polymer, when the crosslinked polymer material is exposed to an aqueous fluid containing the ionically chargeable active agent (e.g. via an Active Agent Concentration Assay), is less than 0.1:1.

Detailed discussion of embodiments of the oral dosage form that are capable of enhancing active agent absorption and bioavailability is provided below.

### Target Tissue

In one embodiment, the oral dosage form is configured to provide delivery of the active agent to a target tissue within the gastrointestinal tract, such as for example the upper gastrointestinal tract or the lower gastrointestinal tract (i.e., the small intestine or large intestine). For example, in one embodiment, the site of delivery of the active agent may be to the mucosa of the small intestine (e.g., the duodenum, jejunum, or ileum) and/or the large intestine (e.g., the ascending colon, the right colic flexure, the transverse colon, the transverse mesocolon, the left colic flexure, the descending colon, the sigmoid colon, and the rectum). In one embodiment, the oral dosage form is configured to provide delivery of the active agent to tissue in the ileum of the small intestine.

According to one embodiment, delivery to a particular region of the gastrointestinal tract, such as to a site in the small intestine, can be achieved by selecting the configuration and composition of the oral dosage form. For example, a protective coating such as an enteric coating can be provided that at least partially shields the dosage form during transit through the stomach and/or other areas of the upper gastrointestinal tract, until a predetermined location in the lower gastrointestinal tract is reached. Further discussion of embodiments of a protectively coated and/or enterically coated dosage form and/or other forms capable of delivering an active agent to a predetermined location in the gastrointestinal tract is provided in further detail below.

### Ionically Chargeable Particulate Hydrogel

The pharmaceutically acceptable oral dosage form for delivery of an active agent to an intestinal site, according to embodiments of the present disclosure, may be capable of providing active agent into close contact with and/or in the vicinity of intestinal tissue at the target intestinal site, and increasing local drug concentration, to promote uptake of the active agent at the target site. As describe above, one potential mechanism of drug exclusion from the hydrogel is by ionic interactions, via Donnan Equilibrium or Donnan Exclusion Effect. For example, in order to maintain negative charges inside of a hydrogel for drug delivery, chemical moieties can be provided that retain negative charge when the hydrogel is in the small intestine. According to some embodiments, the pharmaceutically acceptable oral dosage form may be one suitable for an intestinal pH in a range of from about 4 to about 8. In addition, since the pH of the small intestine can typically range from pH 5 to pH 8, according to certain embodiments it is desirable to use functional groups with a pKa well below this range in the intestinal environment. Carboxylic acids, such as those obtained from crosslinked sodium polyacrylate, will have some negative charges at pH 5-8. However, since the pKa of a carboxylic acid is just below 5 (4.76 for acetic acid), they may not all be in the charged form at pH 5 or 6.

Additionally, the pKa values inside a hydrogel (versus at the surface) may be shifted to somewhat higher pKa values due to charge-charge repulsion inside the hydrogel, making it harder to a mass a large number of charges. This effect has been observed for other hydrogels, for example, crosslinked polyallylamine, where the pKa of the amines (pKa˜9) is shifted by about 1-2 pH units in a hydrogel compared to ordinary dissolved primary amines before polymerization (propylamine pKa˜10.7).

For this reason, in certain embodiments, it may be desirable to have the pKa of the acid groups in the hydrogel well below pKa of 5 in the intestinal environment. Functional groups that fit this criterion include, in certain embodiments, sulfonates and sulfates, with pKa values in the −5 to +2 range. Published values show the pKa of these moieties to be: methane sulfonic acid (−1.92), ethanesulfonic acid (−1.68), benzene sulfonic acid (−2.8), and methanesulfate (−3.4). Consequently, sulfonates and sulfates are preferred functional groups for anionic hydrogels designed to exclude anionic drugs in the small intestine. For practical purposes, the pKa of functional groups as described herein is that as measured in deionized water, as the pKa of such functional groups in intestinal fluid or simulated intestinal fluid (e.g. FASSIF) would be expected to be very similar and substantially the same as the pKa as measured in deionized water.

According to certain embodiments, chargeable moieties comprising sulfates or sulfonates may be preferred to generate negative charge in the hydrogel, as compared to chargeable moieties comprising carboxylates, due to their reduced tendency to strongly bind calcium or magnesium from the small intestinal fluid, essentially reducing the net negative charge inside the hydrogel. According to certain embodiments, for similar reasons of providing a low pKa, it may be desirable to use phosphonates or phosphates to generate negative charge in hydrogels, since certain phosphonates and/or phosphates may have pKa values below 2. According to yet another embodiment, carboxylates may be provided, and/or a combination of different chargeable moieties, such as sulfates and/or sulfonates combined with carboxylates, may be provided.

Examples of suitable anionic hydrogels include those containing the monomers acrylic acid, methacrylic acid, vinyl sulfonic acid, vinyl sulfate, itaconic acid, vinyl phosphate, styrene sulfonic acid, carboxymethyl cellulose, carboxymethyl starch, alginic acid, aspartic acid, glutamic acid, and pharmaceutically acceptable salts thereof.

Examples of suitable anionic hydrogels include poly(acrylic acid), sodium polyacrylate, potassium polyacrylate, mixed sodium and potassium polyacrylate, polymethacrylic acid, sodium polymethacrylate, sodium polyvinylsulfonate, sodium poly(vinyl sulfate), sodium poly(itaconate), sodium polyvinyl phosphate, sodium polystyrene sulfonate, sodium carboxymethyl cellulose, carboxymethyl cellulose, sodium carboxymethyl starch, sodium alginate, sodium polyaspartate, polyaspartic acid, sodium polyglutamate, polyglutamic acid, and copolymers thereof.

In some embodiments, it is preferred to have the polymer contain as much negative charge as possible to better exclude the negatively charged drug molecules. Examples of polymers with chargeable moieties having very high negative charge in the pH 5-8 range include sodium polyacrylate, sodium poly vinylsulfonate, and sodium polyvinylsulfate. Sodium polyvinylsulfonate and sodium polyvinylsulfate are most preferred in this regard due to the very low pKa of the acidic moiety (the sulfonate and sulfate groups) to ensure that as many of the groups are in the charged form as possible at the required pH of 5-8.

In some embodiments, it is preferred to have the polymer be net neutral toward delivery and excretion of sodium and potassium. For example, a polymer dosed to a patient as sodium polyacrylate and excreted as a mixture of sodium and potassium polyacrylate may cause a net excretion of potassium, which may be undesirable. A certain ratio of sodium to potassium in the dosed polymer will result in no substantial excretion of either sodium or potassium.

In some embodiments, the crosslinked polymer material comprising the ionically chargeable hydrogel is capable of taking up a significant amount of fluid in the intestine, to enhance delivery of the active agent. For example, in certain embodiments, the crosslinked polymer material contained in the ionically chargeable hydrogel has a Swelling Ratio of at least at least 5, at least 10, at least 20, at least 25, at least 30, at least 40, at least 50, at least 60, at least 75, at least 85, at least 100, at least 110, at least 120 and, even up to 200 or higher as measured in deionized water. In other embodiments, the crosslinked polymer material contained in the ionically chargeable hydrogel has a Swelling Ratio of at least at least 5, at least 10, at least 20, at least 25, at least 30, at least 35, at least 40, and even up to 100 or higher as measured FASSIF. In some embodiments, the crosslinked polymer material swells at least 100%, at least 200%, at least 500%, at least 1000%, at least 2000%, at least 3000%, at least 4000%, at least 5000%, at least 8000%, at least 10,000% and/or at least 15,000% by weight in deionized water. In some embodiments, the crosslinked polymer material swells at least 100%, at least 200%, at least 500%, at least 1000%, at least 2000%, at least 3000%, at least 4000%, at least 5000%, at least 8000%, at least 10,000% and/or at least 15,000% in FASSIF. For example, in certain embodiments, the crosslinked polymer material absorbs at least 2 mL, at least 3 mL, at least 4 mL, at least 5 mL, at least 8 mL, at least 10 mL, at least 15 mL, at least 20 mL, at least 50 mL, at least 100 mL, at least 125 mL, a least 150 mL, at least 175 mL, and/or at least 200 ml of deionized water.

In some embodiments, the ionically chargeable hydrogel material comprises a plurality of ionically chargeable functional groups. In certain embodiments, the plurality of ionically chargeable functional groups can have either a net cationic charge or a net anionic charge at the intestinal pH. Exemplary ionically chargeable functional groups can be selected from but not limited to the group consisting of sulfonate groups, sulfate groups, carboxylate groups, phosphate groups, and combinations thereof.

To formulate the dosage form described herein, in some embodiments, the dosage form may comprise at least 50 mg, at least 100 mg, at least 200 mg, at least 300 mg, or at least 450 mg of the crosslinked polymer material, and less than 1 g of the crosslinked polymer material. In certain embodiments, the crosslinked polymer material comprises at least 10%, at least 20%, at least 30%, at least 40%, at least 50%, at least 60%, at least 70%, or at least 90% of the dosage form by weight. In some embodiments, a ratio of the mass of the crosslinked polymer material in the dosage form to the mass of the ionically chargeable active agent is at least 2:1, at least 4:1, at least 8:1, at least 10:1, at least 20:1, at least 50:1, or at least 100:1.

In one embodiment, the crosslinked polymer material contains an ionically chargeable moiety on at least 10%, at least 15%, at least 20%, at least 30%, at least 50%, at least 75%, at least 80%, at least 85%, at least 90%, or up to 99% of the monomers comprising the polymer. In certain embodiments, the crosslinked polymer material contains a negatively chargeable moiety on at least 10%, at least 15%, at least 20%, at least 30%, at least 50%, at least 75%, at least 80%, at least 85%, at least 90%, or up to 99% of the monomers comprising the polymer. In certain embodiments, the crosslinked polymer material contains a positively chargeable moiety on at least 10%, at least 15%, at least 20%, at least 30%, at least 50%, at least 75%, at least 80%, at least 85%, at least 90%, or up to 99% of the monomers comprising the polymer. For example, in one embodiment, the crosslinked polymer material contains a carboxylate moiety on at least 10%, at least 15%, at least 20%, at least 30%, at least 50%, at least 75%, at least 80%, at least 85%, at least 90%, or up to 99% of the monomers comprising the polymer. In another embodiment, the crosslinked polymer material contains a phosphate moiety on at least 10%, at least 15%, at least 20%, at least 30%, at least 50%, at least 75%, at least 80%, at least 85%, at least 90%, or up to 99% of the monomers comprising the polymer. In yet another embodiment, the crosslinked polymer material contains a sulfonate or sulfate moiety on at least 10%, at least 15%, at least 20%, at least 30%, at least 50%, at least 75%, at least 80%, at least 85%, at least 90%, or up to 99% of the monomers comprising the polymer. In further embodiments, a sulfur atom comprises at least 2%, at least 4%, at least 8%, at least 10%, or at least 16% of the dry weight of the crosslinked polymer.

In some embodiments, the crosslinked polymer is formed from residues comprising one or more vinyl-substituted amide moieties, such as for example a residue of 2-acrylamido-2-methylpropane sulfonic acid or its pharmaceutically acceptable salt, 4-vinylbenzenesulfonic acid or its pharmaceutical salt (e.g. styrene sulfonate), and/or a residue of methylenebisacrylamide or its pharmaceutically acceptable salt. In some embodiments, at least 10%, at least 15%, at least 20%, at least 30%, at least 45%, at least 50%, at least 60%, at least 75%, or at least 80% by weight of the crosslinked polymer comprises a residue of 2-acrylamido-2-methylpropane sulfonic acid or its pharmaceutically acceptable salt. In some embodiments, no more than 90%, 80%, 70%, 60%, 55%, 50%, 45%, 30% or 20% by weight of the crosslinked polymer material comprises the residue of 2-acrylamido-2-methylpropane sulfonic acid or its pharmaceutically acceptable salt. In some embodiments, at least 10%, at least 15%, at least 20%, at least 30%, at least 45%, at least 50%, at least 60%, at least 75%, or at least 80% by weight of the crosslinked polymer material comprises a residue of 4-vinyl benzenesulfonic acid or its pharmaceutically acceptable salt. In some embodiments, no more than 90%, 80%, 70%, 60%, 55%, 50%, 45%, 30% or 20% by weight of the crosslinked polymer material comprises the residue of 4-vinyl benzenesulfonate or its pharmaceutically acceptable salt. In some embodiments, from 0.2 wt % to 20 wt % of the crosslinked polymer comprises a crosslinker comprising a residue of methylenebisacrylamide or its pharmaceutically acceptable salt. In some embodiments, from 1 wt % to 5 wt % of crosslinked polymer comprises a residue of methylenebisacrylamide or its pharmaceutically acceptable salt. In certain embodiments, 2 wt % of crosslinked polymer comprises a residue of methylenebisacrylamide or its pharmaceutically acceptable salt. In certain embodiments, the pharmaceutically acceptable salt can be sodium salts, such as in sodium styrene sulfonate and sodium 2-acrylamido-2-methylpropane sulfonate, but other pharmaceutically acceptable salt forms may also be provided.

According to certain embodiments of the present disclosure, a hydrogel is provided that comprises an ionically chargeable crosslinked polymer material comprising (i) a residue of 4-vinylbenzenesulfonate or its pharmaceutically acceptable salt, (i) a residue of 2-acrylamido-2-methylpropane sulfonic acid or its pharmaceutically acceptable salt, and (iii) a crosslinker comprising a residue of methylenebisacrylamide or its pharmaceutically acceptable salt. According to certain embodiments, a ratio by weight of the residue of 4-vinylbenzenesulfonate or its pharmaceutically acceptable salt to the residue of 2-acrylamido-2-methylpropane sulfonic acid or its pharmaceutically acceptable salt in the ionically chargeable crosslinked polymer is within a range of 0.25:1 to 1:0.25, such as within a range of 0.5:1 to 1:0.5 and/or 0.75:1 to 1:0.75. The hydrogel may, in certain embodiments, be in the form of hydrogel particulates.

In some embodiments, the ionically chargeable cross-linked polymer comprises carboxylate and/or carboxylic acid groups. In some embodiments, the hydrogel particulates, before administration to the patient, contain less than 10% by weight of the ionically chargeable active agent within an internal dosage form volume occupied by the hydrogel particulates. In certain embodiments, the hydrogel particulates have a mesh size in the range of 0.05 mm to 2 mm, and/or 0.5 mm to 2 mm. According to one embodiment, the hydrogel particulates have a mesh size in the range of 0.05 mm to 2 mm, and/or 0.5 mm to 2 mm.

In some embodiments, the crosslinked polymer material comprises negatively chargeable functional groups having a pKa of less than 5 as in deionized water at a pH of about 7. In some embodiments, the crosslinked polymer material comprises negatively chargeable functional groups having a pKa of less than 3 as in deionized water at a pH of about 7. In some embodiments, at least 10%, at least 15%, at least 20%, at least 30%, at least 50%, at least 75%, at least 80%, at least 85%, at least 90%, or up to 99% of the monomers of the crosslinked polymer material comprise negatively chargeable functional groups having a pKa of less than 5 in deionized water at a pH of about 7. In some embodiments, at least 10%%, at least 15%, at least 20%, at least 30%, at least 50%, at least 75%, at least 80%, at least 85%, at least 90%, or up to 99% of the monomers of the crosslinked polymer material comprise negatively chargeable functional groups having a pKa of less than 3 in deionized water at a pH of about 7.

### Active Agent

The oral dosage form according to embodiments of the present disclosure is adapted to deliver any of a wide range of active agents to a tissue site. Thus, for example, the oral dosage form may be adapted to deliver a single active agent or multiple active agents (e.g., two, three or more active agents, either serially or simultaneously) to the tissue site. Additionally, the active agents may be in any of a wide range of alternative forms such as pharmaceutically acceptable salt forms, free acid forms, free base forms, and hydrates.

In general, the active agent may be in particulate, liquid, or gel form and may comprise any of a range of compositions having biological relevance, e.g., metals, metal oxides, peptides, peptides structurally engineered to resist enzymatic degradation, antibodies, hormones, enzymes, growth factors, small organic molecules, ligands, or other pharmaceuticals, nutraceuticals, or biologics. In some embodiments, the agent(s) may include one or more large molecules (e.g., proteins and/or protein conjugates), and/or one or more small molecules (e.g., small organic molecules, and/or small peptides) as the agent(s). In one exemplary embodiment, the active agent comprises at least one polypeptide and/or small molecule having a therapeutic treatment effect. Examples of active agents that can be delivered by the oral dosage form can include at least one of Insulin, Vasopressin, Calcitonin (including salmon calcitonin), Oxytocin, Adrenocorticotropic hormone (ACTH), Leuprorelin, Octreotide, Ixazomib, Adlyxin. Abaloparatide, Abaloparatide, Angiotensin II, Etelcalcetide, Macimorelin, Plecanatide, Semaglutide and other GLP-1 analogs (GLP-1 agonists), ¹⁷⁷Lu DOTA-TATE (Lutathera®), ⁶⁸Ga DOTA-TOC, Afamelanotide, Bremelanotide, Enfortumab Vedotin-Ejfv, Polatuzumab Vedotin-Piiq, setmelanotide, BULEVIRTIDE, PConsensus, laminin-derived peptide, nucleosomal peptides, DWEYS peptide, glatiramer acetate, thymopentin-5, peptide P140, AMG623, Bivalirudin, Buserelin, Corticotropin, Cosyntropin, Enfuvirtide, Eptifibatide, Gramicidin D, Lepirudin, Leuprolide, Lucinactant, Nesiritide, Oxytocin, Pramlintide, Secretin, Sermorelin, Teduglutide, Thymalfasi, Rs-ARF2, Iturins, Histatin 5, Aureobasidins, Echinocandins, Pneumocandins, aculeacins (A-D, F), mulundocandins, WF11899 (A, B, and C), WmKT, Cilofungin, anidulafungin, FOL-005, Teriparatide, ZPGG-72, ZP3022, MOD-6030, ZP2929, HM12525A, VSR859, NN9926, TTP273/TTP054, ZYOG1, MAR709, TT401, HM11260C, PB1023, ITCA, parathyroid hormone (PTH), teriparatide (a recombinant form of PTH), peptide agonists of GLP-1, such as exenatide, liraglutide, lixisenatide, albiglutide, dulaglutide, GLP-1/GIP co-agonists, such as tirzepatide, GLP-2 agonists and peptide GPCR agonists. Additional examples of active agents include antibiotics such as azithromycin, vancomycin, dalbavancin (Dalvance), micafungin (Mycamine), Brilicidin, Avidocin, Purocin, and Arenicin. Active agents may also include the antimycobacterial agents clofazimine, ethionamide, para-aminosalicylic acid, and Amikacin.

In yet another embodiment, the active agent can comprise other large molecules and/or other structures other than those specifically listed above, such as for example any one or more of antibodies (monoclonal and polyclonal) or antibody fragments, polysaccharides, carbohydrates, nanoparticles, vaccines, biologics, nucleic acids, cells and cell therapies, DNA, RNA, siRNA, blood factors, gene therapies, thrombolytic agents (tissue plasminogen activator), growth factors (erythropoietin), interferons, interleukin-based molecules, fusion proteins, recombinant proteins, therapeutic enzymes, and others. The active agent may also and/or alternatively comprise at least one of a small molecule drug, a drug conjugate, a prodrug, a small organic molecule (e.g., with a molecular weight of about 500 Da or less), a metabolically activated agent (e.g., a metabolite), a nutrient, a supplement, and the like.

According to one embodiment, the oral dosage form disclosed herein is capable of providing improved bioavailability in delivering an active agent that may be otherwise incompletely absorbed in the intestine. For example, the oral dosage form disclosed herein can be capable of providing surprisingly improved bioavailability for polypeptides and/or other small molecules having a relatively high molecular weight, which agents may be otherwise difficult to effectively administer due to their relatively large size. Examples of such active agents may include polypeptides and/or small molecules having a size of at least about 450 Da. However, according to one embodiment, the molecular weight of the active agent may still be below about 200,000 Da, to allow for good delivery/absorption of the active agent in the intestine. According to one example, in one embodiment the active agent has a molecular weight of at least about 2000 Da. By way of further example, in one embodiment the active agent has a molecular weight of at least about 5000 Da. By way of yet a further example, in one embodiment the active agent has a molecular weight of at least about 10,000 Da. While the active agent according to one embodiment will generally have a molecular weight below about 600,000 Da, as has been described above, the molecular weight may also in one example be below about 200,000 Da, such as below about 100,000 Da. For example, the active agent provided as a part of the oral dosage form may have a molecular weight in one embodiment that is in the range of from about 450 Da to about 500,000 Da, such as about 450 Da to about 25,000 Da, and even 450 Da to 10,000 Da, such as about 450 Da to about 6000 Da. For example, in one embodiment the active agent may have a molecular weight in a range of from about 1000 Da to about 25,000 Da, and even about 1,000 Da to about 10,000 Da, such as about 1000 Da to 5000 Da. As previously noted, the oral dosage form may contain two or more agents independently selected from molecules having a molecular weight within the ranges recited herein.

As described above, to enhance the ionic interaction, the active agent can one that is ionically charged in the intestinal environment. For example, in one embodiment, the ionically chargeable active agent comprises a plurality of ionically chargeable groups, such as at least one, at least two, at least three, at least four, at least five, and even more ionically chargeable groups that are expected to be negatively or positively charged at intestinal pH (e.g., at a pH of 5-8). To maximize the repulsion, in some embodiments, the ionically chargeable hydrogel material and the ionically chargeable active agent both have a net cationic charge at the intestinal pH. Alternatively, in other embodiments, the ionically chargeable hydrogel material and the ionically chargeable active agent both have a net anionic charge at the intestinal pH.

In another embodiment, the ionically chargeable active agent is at least one of a peptide or modified peptide, and polynucleotide, having a molecular weight of at least 500 g/mol, at least 1000 g/mol, at least 2000 g/mol, at least 4000 g/mol, at least 500 g/mol, and/or up to 10,000 g/mol.

The dosage form disclosed herein can significantly increase the local concentration of the drug, for example, in one embodiment, when the dosage form is dispersed in water, the concentration of the ionically chargeable active agent 10 minutes after dispersal in water is at least 20% higher than when the same dosage form without the crosslinked polymer is dispersed in water for the same time period. In another embodiment, when the dosage form is dispersed in water, the concentration of the ionically chargeable active agent 30 minutes after dispersal in water is at least 50% higher than when the same dosage form without the crosslinked polymer is dispersed in water for the same time period. In yet another embodiment, when the dosage form is dispersed in water, the concentration of the ionically chargeable active agent 60 minutes after dispersal in water is at least 100% higher than when the same dosage form without the crosslinked polymer is dispersed in water for the same time period. In still another embodiment, when the dosage form is dispersed in water, the concentration of the ionically chargeable active agent 90 minutes after dispersal in water is at least 200% higher than when the same dosage form without the crosslinked polymer is dispersed in water for the same time period. In further embodiment, when the dosage form is dispersed in water, the concentration of the ionically chargeable active agent 120 minutes after dispersal in water is up to 300% higher than when the same dosage form without the crosslinked polymer is dispersed in water for the same time period.

Given the varying acidities in different sections of the GI tract, it is beneficial for the ionically chargeable hydrogel and the ionically chargeable active agent to have net ionic charges with the same sign at different pHs. In some embodiments, the crosslinked polymer material and the ionically chargeable active agent have net ionic charges with the same sign (e.g. either both net negative or both net positive) at pH 2. In some embodiments, the crosslinked polymer material and the ionically chargeable active agent have net ionic charges with the same sign at pH 5. In some embodiments, the crosslinked polymer material and the ionically chargeable active agent have net ionic charges with the same sign at pH 6. In some embodiments, the crosslinked polymer material and the ionically chargeable active agent have net ionic charges with the same sign at pH 7.

### Permeation Enhancer

In yet another embodiment, the oral dosage form comprises at least one permeation enhancer to enhance permeation of the active agent through the intestinal tissue. In some embodiments, the permeation enhancer may be capable of opening a tight junction between cells (e.g., intestinal cells or epithelial cells). A permeation enhancer may, in some instances, facilitate uptake of an agent into epithelial cells. Representative classes of permeation enhancers include, but are not limited to, a fatty acid, a medium chain glyceride, a surfactant, a steroidal detergent, an acyl carnitine, lauroyl carnitine, palmitoyl carnitine, an alkanoyl choline, an N-acetylated amino acid, esters, salts, bile salts, sodium salts, nitrogen-containing rings, derivatives thereof, and combinations thereof. The permeation enhancer may be anionic, cationic, zwitterionic, or nonionic. Anionic permeation enhancers include, but are not limited to, sodium lauryl sulfate, sodium decyl sulfate, sodium octyl sulfate, N-lauryl sarcosinate, and sodium carparate. Cationic permeation enhancers include, but are not limited to, cetyltrimethyl ammonium bromide, decyltrimethyl ammonium bromide, benzyldimethyldodecyl ammonium chloride, myristyltrimethylammonium chloride, and dodecylpyridinium chloride. Zwitterionic permeation enhancers include, but are not limited to, N-dodecyl-N,N-dimethyl-3-ammonio-1-propanesulfonate, 3-(N,N-dimethylpalmitylammonio)propanesulfonate. Fatty acids include, but are not limited to, butyric, caproic, caprylic, pelargonic, capric, lauric, myristic, palmitic, stearic, arachidic, oleic, linoleic, and linolinic acid, salts thereof, derivatives thereof, and combinations thereof. In some embodiments, a fatty acid may be modified as an ester, for example, a glyceride, a monoglyceride, a diglyceride, or a triglyceride. Bile acids or salts including conjugated or unconjugated bile acid permeation enhancers include, but are not limited to, cholate, deoxycholate, tauro-cholate, glycocholate, taurodexycholate, ursodeoxycholate, tauroursodeoxycholate, chenodeoxycholate, derivatives thereof, salts thereof, and combinations thereof. In some embodiments, permeation enhancers include a metal chelator, such as EDTA or EGTA, a surfactant such as sodium dodecyl sulfate, polyethylene ethers or esters, polyethylene glycol-12 lauryl ether, salicylate polysorbate 80, nonylphenoxypolyoxyethylene, dioctyl sodium sulfosuccinate, saponin, palmitoyl carnitine, lauroyl-I-carnitine, dodecyl maltoside, acyl carnitines, alkanoyl cjolline, and combinations thereof. Other permeation enhancers include, but are not limited to, 3-nitrobenzoate, zoonula occulden toxin, fatty acid ester of lactic acid salts, glycyrrhizic acid salt, hydroxyl beta-cyclodextrin, N-acetylated amino acids such as sodium N-[8-(2-hydroxybenzoyl)amino]caprylate and chitosan, micelle forming agents, passageway forming agents, agents that modify the micelle forming agent, agents that modify the passageway forming agents, salts thereof, derivatives thereof, and combinations thereof. In some embodiments, micelle forming agents include bile salts. In some embodiments, passageway-forming agents include antimicrobial peptides. In some embodiments, agents that modify the micelle forming agents include agents that change the critical micelle concentration of the micelle forming agents. An exemplary permeation enhancer is 1% by weight 3-(N,N-dimethylpalmitylammonio)propanesulfonate. Permeation enhancers are also described in patent application publication US 2013/0274352, the contents of which are incorporated in their entirety herein. In one embodiment, the permeation enhancers can comprise at least one of EDTA, palmitoyl carnitine, lauroyl carnitine, dimethyl palmitoyl ammonio propanesulfonate (PPS), and sodium caprate.

In one embodiment, permeation enhancers selected for the oral dosage form may be selected on the basis of one or more of the predominant permeation mechanism and the hydrophilicity and/or hydrophobicity of the permeation enhancer. For example, permeation enhancers that are fatty esters and/or permeation enhancers having nitrogen-containing rings may exhibit more paracellular transport activity, whereas cationic and zwitterionic permeation enhancers may exhibit more transcellular activity, as described for example in the article to Whitehead and Mitragotri entitled “Mechanistic Analysis of Chemical Permeation Enhancers for Oral Drug Delivery” in Pharmaceutical Research, Vol. 25, No. 6, June 2008, pages 1412-1419, which is hereby incorporated by reference herein in its entirety. Furthermore, for those permeation enhancers having a transcellular mechanism, increases in hydrophobicity of the permeation enhancer may enhance this mechanism, whereas for permeation enhancers having more paracellular transport activity, greater enhancement may be seen for those permeation enhancers that are more hydrophillic (such as by interacting with hydrophilic constituents of tight junctions). In one embodiment the relative hydrophobicity/hydrophilicity of the enhancer may be determined by its log P value, with P being the octanol/water partition coefficient for the compound. For example, in one embodiment, to enhance transcellular transport, a permeation enhancer may have a log P value of at least 2, such as at least 4, and even at least 6. Conversely, to enhance paracellular transport, a permeation enhancer may in one embodiment have a log P of less than about 4, such as less than 2, and even less than 0.

A content of the permeation enhancer in the oral dosage form in one embodiment may be at least about 0.01% by weight, such as at least about 0.1% by weight, and no more than about 80% by weight, and may even be less than about 30% by weight. For example, in one embodiment, the content of permeation enhancer in the oral dosage form may be at least about 0.01% by weight, such as at least about 0.1% by weight, including at least about 1% by weight, such as at least about 5% by weight, and even at least about 10% by weight, such as at least about 30% by weight, or even at least about 50% by weight, such as at least about 70% by weight. For example, in one embodiment, the content of permeation enhancer may be in the range of from 0.1% by weight to 70% by weight, such as from about 0.1% by weight to about 20% by weight, and even from about 1% by weight to about 10% by weight.

As described above, in certain embodiment, ionic interaction (repulsion) can play an important role in the dosage form disclosed herein. In certain embodiments, the same theory relates to permeation enhancers used. Anionic permeation enhancers (such as sodium caprate) as well as anionic micelles of such enhancers will potentially be excluded from the hydrogel for the same reasons as described above (anionic charge and size/diffusion coefficient). Zwiterionic permeation enhancers, such as dimethyl palmitoyl ammonio propanesulfonate (PPS) may also be excluded by the size/diffusion coefficient effect, and by charge effects if they form micelles where the outmost surface of the micelle is anionic. Like the ionically chargeable hydrogel and active agent, the permeation enhancer can also be ionically charged by having, for example, one or more ionically chargeable groups. In some embodiments, the crosslinked polymer material, the ionically chargeable active agent, and the permeation enhancer all have the same net ionic charge at the intestinal pH in a range of 4 to 8. In certain embodiments, the crosslinked polymer material, the ionically chargeable active agent, and the permeation enhancer all have a net negative charge at an intestinal pH in a range of 4 to 8. In some embodiments, the crosslinked polymer material, the ionically chargeable active agent, and the permeation enhancer all have a net negative charge in aqueous solution at pH of about 7. In some embodiments, the crosslinked polymer material, the ionically chargeable active agent, and the permeation enhancer all have a net negative charge in aqueous solution at pH of about 5.

### Other Additives

The oral dosage form can comprise further additives in addition to the active agent, particulate hydrogel and optional permeation enhancer.

In one embodiment, the oral dosage form may comprise an osmagent that assists in delivery of the active agent. Without being limited by any one theory, it is believed that the osmagent may assist in expelling the active agent from the oral dosage form, by absorbing water and pushing the active agent from the oral dosage form, and/or may help to open tight junctions in the intestine by pulling water therefrom. In one embodiment, an osmagent capable of being hydrated may include water-soluble salts, carbohydrates, small molecules, amino acids, and combinations thereof. Exemplary water-soluble salts may include, without limitation, magnesium chloride, magnesium sulfate, lithium chloride, sodium chloride, potassium chloride, lithium sulfate, sodium sulfate, potassium sulfate, sodium hydrogen phosphate, potassium hydrogen phosphate, sodium acetate, potassium acetate, magnesium succinate, sodium benzoate, sodium citrate, sodium ascorbate, and the like, and combinations thereof. Exemplary carbohydrates may include sugars such as arabinose, ribose, xylose, glucose, fructose, galactose, mannose, sucrose, maltose, lactose, raffinose, and the like, and combinations thereof. Exemplary amino acids may include glycine, leucine, alanine, methionine, and the like, and combinations thereof. In one embodiment, the osmagent provided in the oral dosage form comprises at least one of sucrose, mannitol, fructose and polyethylene glycol. A content of the osmagent in the oral dosage form in one embodiment may be at least about 1% by weight, and less than about 60% by weight, such as from about 10% by weight to about 50% by weight, and even from about 20% by weight to about 40% by weight.

In another embodiment, the oral dosage form may comprise a protease inhibitor. As used herein, a “protease inhibitor” or “antiprotease” refers to a molecule (usually a protein) that inhibits the function of proteases (enzymes that aid the breakdown of proteins). In the present disclosure, the protease inhibitor is to inhibit breakdown of active agent peptides during, e.g., its delivery in the intestine. Exemplary protease inhibitors include aspartic protease inhibitors, cysteine protease inhibitors, metalloprotease inhibitors, serine protease inhibitors, threonine protease inhibitors, and trypsin inhibitors. In one embodiment of the present disclosure, the oral dosage form comprises a soybean trypsin inhibitor.

Other additives and/or excipients that can be provided as a part of the oral dosage form can include one or more of stabilizers, glidants, bulking agents, anti-adherents, mucoadhesive agents, binders, sorbents, preservatives, cryoprotectants, hydrating agents, enzyme inhibitors, mucus modifying agents (e.g., mucus drying agents, etc.), pH modifying agents, solubilizers, plasticizers, crystallization inhibitors, bulk filling agents, bioavailability enhancers, and combinations thereof. In some embodiments, the additives and/or excipients may include polyethylene glycols, polyethylene oxides, humectants, vegetable oils, medium chain mono, di-, and triglycerides, lecithin, waxes, hydrogenated vegetable oils, colloidal silicon dioxide, polyvinylpyrrolidone (PVP) (“povidone”), celluloses, CARBOPOL® polymers (Lubrizol Advanced Materials, Inc.) (i.e., crosslinked acrylic acid-based polymers), acrylate polymers, pectin, sugars, magnesium sulfate, or other hydrogel forming polymers.

### Protective Coating

The oral dosage form according to one embodiment further comprises a protective coating that at least partially protects the oral dosage form from the acidic environment in the stomach to deliver the active agent to a region of the intestine. The protective coating can, in one embodiment, form an outer coating of the oral dosage form that protects the active agent and/or the particulate hydrogel, or other additives inside the oral dosage form. While in one embodiment the protective coating completely covers an outer surface of the delivery structure comprising the hydrogel particulate and active agent of the dosage form, the protective coating may also optionally be devised to cover only a portion of the outer surface of the delivery structure. The protective coating can also comprise only a single coating layer, or can be configured as multiple coating layers. For example, in one embodiment, the protective coating comprises a capsule form. In another embodiment, the protective coating comprises a tablet form. In yet another embodiment, the protective coating comprises an enteric coating to release the ionically chargeable active agent in the small intestine. In still another embodiment, the protective coating comprises a time-release coating. In a further embodiment, the protective coating comprises a capsule coated with an enteric coating.

According to one embodiment, the protective coating may be an enteric coating that is a pH dependent coating, having an enteric material that is a polymer that is substantially insoluble in the acidic environment of the stomach, but that has increased solubility in intestinal fluids that are at a higher pH. That is, the enteric coating may preferentially dissolve and/or become at least partially permeable in the intestine as opposed to in the stomach. Thus, in one embodiment, the protective coating comprises an enteric coating to release the ionically chargeable active agent in the small intestine. For example, the enteric coating may be formed of an enteric material that is substantially insoluble at a pH below about 5, such as in the acidic environment of the stomach, but that becomes soluble at higher pH, such as a pH of at least about 5.5 for the duodenum, a pH of at least about 6.5 for the jejunum, and a pH of at least about 7.0, such as at least about 7.5 for the ileum (the duodenum, jejunum and ileum are part of the small intestine). That is, the enteric coating can be selected to be insoluble at lower pH, but soluble at a higher pH, such that the enteric coating can be made to dissolve and/or become at least partially permeable and release the contents of the oral dosage form once an environment of the gastrointestinal system is reached having a pH in which the material of the enteric coating is soluble. Accordingly, suitable enteric materials for forming the enteric coating in one embodiment are those that are not soluble until a pH of at least about 5.5 is reached, such as a pH of at least about 6.0. In one embodiment, suitable enteric materials for forming the enteric coating in one embodiment are those that are not soluble until a pH of at least about 6.5 is reached, such as a pH of at least about 7.0, and even a pH of at least about 7.5. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, poly(vinylalcohol), natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, Kollicoat MAE 100P and Aquateric). For example, in one embodiment the enteric materials used to form the enteric coating can comprise at least one of Eudragit S100 (poly(methacrylic acid-co-methyl methacrylate) 1:2), Eudragit L100 (poly(methacrylic acid-co-methyl methacrylate) 1:1), and Kollicoat MAE 100P (methacrylic acid ethyl acrylate copolymer 1:1). The solubility of each of the above materials at a specific pH is either known or is readily determinable in vitro. For example, the foregoing is a list of possible materials, but one of skill in the art with the benefit of the instant disclosure would recognize that the foregoing list is not comprehensive and that there are other enteric materials that may be used. In yet another embodiment, the protective coating may be one that dissolved and/or becomes partially permeable due to a change in environment that is unrelated to pH. Furthermore, in another embodiment, the protective and/or enteric coating may be one that dissolves and/or becomes at least partially permeable at a predetermined rate as it passes through the gastrointestinal system, to provide a controlled and/or timed release of the active agent at a predetermined region of the intestine.

In one embodiment, the protective coating comprises at least a portion thereof that becomes permeable and/or dissolves under predetermined conditions, such as at a predetermined pH (e.g., a pH at a targeted site of the intestine), or following exposure to fluid for a pre-determined period of time (e.g., controlled release following administration at a predetermined point in time). In one embodiment, the protective coating substantially entirely comprises a coating of a material that becomes permeable and/or dissolved under the predetermined conditions.

In one embodiment, by providing a protective coating having a permeable and/or dissolving portion that surrounds a majority of the surface of the dosage form, the contents of the dosage form can be effectively released, and in a multi-directional manner, without unnecessarily retaining contents inside the dosage form. Furthermore, in yet another embodiment, by providing the permeable and/or dissolving portion about a majority of at least the surface of a region of the dosage form containing the active agent delivery region(s), good release of the particulate hydrogel and active agent delivery regions from a relatively large surface region of the dosage form can be provided.

The protective coating is formed on the surface of the delivery structure according to a suitable method. In one embodiment, the protective coating is formed by spray coating materials such as enteric materials onto the surface of the delivery structure, until a coating having a thickness within a predetermined range has been formed. The protective material may, in one embodiment, be sprayed relatively uniformly on the delivery structure to provide a protective coating having a uniform thickness on the surface of the oral dosage form. The protective coating may also, in another embodiment, be sprayed non-uniformly, according to a configuration of the oral dosage form and the desired release characteristics. In yet another embodiment, the protective coating can be formed on the surface of the delivery structure by a dip-coating method, where the surface of the oral dosage form is dipped or otherwise immersed in a fluid containing the protective coating materials, such as enteric coating materials, to form a coating of the protective materials on the surface.

In some embodiments, the oral dosage form may be configured for controlled release (time release) of the active agent at a region in the intestine, for example by providing a protective coating corresponding to an enteric coating that provides for controlled release at a predetermined pH and/or pH range. Additionally and/or alternatively, other ingredients and/or excipients may be provided in the oral dosage form to provide for a controlled release of the active agent and particulate hydrogel.

According to one embodiment, the oral dosage form is provided in a size that provides good delivery of the active agent in the intestinal tract, without excessively occluding or blocking the intestinal tract. For example, the longest dimension of the oral dosage form may be less than about 3 cm, such as less than about 2 cm, and even less than about 1.5 cm. Typically, the longest dimension of the oral dosage form will be in the range of from about 0.5 cm to about 3 cm, such as from about 1 cm to about 3 cm, and even from about 1 cm to about 2 cm. Suitable capsule sizes may be, for example, size 1, 0, 00 and 000, and including the “EL” versions of any of these sizes.

### Method of Treatment

In some embodiments, an oral dosage form may be administered to an individual, patient, or a subject. In some cases, the oral dosage form may be administered as a single dosage. In other embodiments, a plurality of oral dosage forms may be administered to provide multiple dosages overtime. Alternatively, the oral dosage form described herein may be administered to a subject in need thereof without food or under a fasting condition. For example, the oral dosage form may be administered at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, between about 3 hours to about 12 hours, between about 4 hours to about 12 hours, between about 4 hours to about 10 hours, between about 4 hours to about 8 hours, or between about 4 hours to about 6 hours, after consumption of food by a subject.

Alternatively, the oral dosage forms described herein may be administered to a subject in need thereof under a condition of fluid restriction. This restriction shall mean that over the stated time, the subject may consume less than 16 oz. of fluids, less than 8 oz of fluids, less than 4 oz of fluids, less than 2 oz of fluids, or less than 1 oz of fluids. For example, the subject may be restricted in their consumption of fluids prior to being administered the oral dosage form for at least about 1 hours, at least about 2 hours, at least about 3 hours, at least about 4 hours, at least about 5 hours, at least about 8 hours, between about 1 hours to about 2 hours, between about 1 hours to about 4 hours. Additionally, the subject may be restricted in their consumption of fluids after being administered the oral dosage form for at least about 1 hours, at least about 2 hours, at least about 3 hours, at least about 4 hours, at least about 5 hours, at least about 8 hours, between about 1 hours to about 2 hours, between about 1 hours to about 4 hours.

Treatment can be continued for as long or as short of a period as desired. The oral dosage form may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result is achieved. A treatment regimen can include a corrective phase, during which a dose sufficient, for example, to reduce symptoms is administered, and can be followed by a maintenance phase, during which a lower dose sufficient to maintain the reduced symptoms is administered. A suitable maintenance dose is likely to be found in the lower parts of the dose ranges provided herein, but corrective and maintenance doses can readily be established for individual subjects by those of skill in the art without undue experimentation, based on the disclosure herein.

In certain embodiments, the oral dosage form may be used to deliver an agent (e.g., octreotide) to a subject in need thereof. In some embodiments, the oral dosage form may be capable of delivering insulin to a patient in need thereof, such as a person suffering from diabetes. In certain embodiments, the oral dosage form may be used to deliver an agent (e.g., calcitonin) to a subject in need thereof. For example, the oral dosage form may be used to treat hypercalcemia. In another example, the oral dosage form may be used to treat a bone disease, such as osteoporosis. In yet another embodiment, the oral dosage form may be used to treat a mental disorder, such as bipolar disorder or mania. In yet another embodiment the oral dosage form may deliver an active agent such as a GLP-1 agonist and/or GIP/GLP1 co-agonist to treat a disorder such as type II diabetes and/or obesity in a patient in need thereof. In yet another embodiment, the oral dosage form may deliver an active agent such as an enzyme-resistant peptide to treat a disorder such as a metabolic disorder to a patient in need thereof.

For example, in one embodiment of the present disclosure, a method for treating or ameliorating the effect of a condition in a subject is provided. The method comprises administering to the subject an effective amount of the dosage form disclosed herein. In some embodiments, the condition is selected from type 2 diabetes mellitus, obesity, and combinations thereof. In some embodiments, the ionically chargeable active agent in the dosage form comprises a peptide having GIP-1 agonist activity or GIP/GLP1 co-agonist activity. Such peptide can be selected from, e.g., semaglutide, exenatide, liraglutide, lixisenatide, albiglutide, dulaglutide, tirzepatide, and combinations thereof.

The oral dosage forms described herein may be used to administer an agent to patients (e.g., animals and/or humans) in need of such treatment in dosages that will provide optimal pharmaceutical efficacy. It will be appreciated that the number and/or type of oral dosage forms required for use in any particular application will vary from 5 patient to patient, not only with the particular agent selected, but also with the concentration of agent in the oral dosage form, the nature of the condition being treated, the age and condition of the patient, concurrent medication or special diets then being followed by the patient, and other factors which those skilled in the art will recognize, with the appropriate dosage ultimately being at the discretion of the attendant physician.

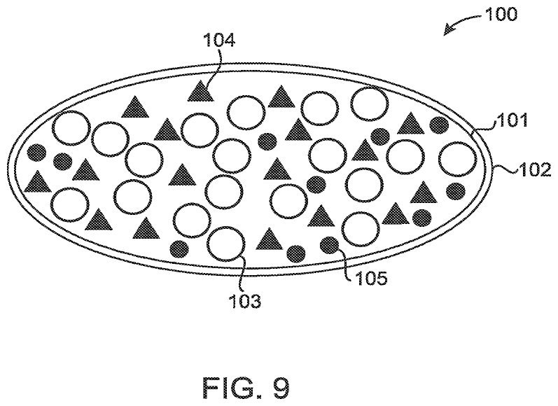

FIG. 9 is a schematic showing an embodiment of an oral dosage form comprising ionically chargeable hydrogel and ionically chargeable active agent. The oral dosage form 100 comprises a delivery device 101, such as a capsule, tablet, or other structure capable of providing the ionically chargeable hydrogel. The delivery device 101 is at least partly covered with a protective coating 102, such as an enteric coating. The delivery device further contains a plurality of hydrogel particulates 103 comprising ionically chargeable hydrogel, and active agent 104, with a permeation enhancer 105 further optionally being provided. Other embodiments of the oral dosage form may also be provided.

According to certain embodiments, and without being limited by any theory, it is believed that engineered hydrogel particulates according to aspects herein selectively absorb water versus active agent, increasing local active agent and/or permeating concentration outside of the hydrogel, which then leads to increased active agent absorption. According to certain embodiments, the ionically chargeable hydrogel repels ionically chargeable active agent and/or permeation enhancer (e.g., as per the Donnan equilibrium). According to further embodiments, and without being limited to any theory, it is believed that the relative lack of macroscopic channels in the hydrogel particulates limits the diffusion of larger molecules through the polymer network, limiting entry of larger active agents into the hydrogel polymer network, such as for example certain peptides. According to yet another embodiment, and without being limited by any theory, it is believed that the hydrogel particulates may reduce protease activity, such as by sequestration and/or self-inactivation of proteases, including those found in the gut.

According to one embodiment, the dosage form or method herein comprises ionically chargeable hydrogel material that reduces the activity of proteases at the intestinal site. For example, the proteases that exhibit a reduction in activity in the presence of the ionically chargeable hydrogel material may be those having a net ionic charge with a sign that is the opposite of a sign of a net ionic charge of the ionically chargeable hydrogel material at an intestinal pH in a range of from about 4 to about 8. Accordingly, in certain embodiments, the dosage form comprising the ionically chargeable hydrogel material may comprise an ionically chargeable hydrogel material with a net ionic charge with a sign that is the opposite of a sign of a net ionic charge of the protease at an intestinal pH in a range of from about 4 to about 8, to reduce the activity of the one or more proteases and inhibit degradation of the active agent to enhance delivery thereof. A method of delivering an active agent with the ionically chargeable hydrogel material as a part of the dosage form may, in certain embodiments, comprise providing an ionically chargeable hydrogel material that is capable of reducing the activity of proteases that tend to degrade the active agent at the intestine. According to one embodiment, the dosage form or method provides a delivery device with ionically chargeable hydrogel material that reduces the activity of any of the proteases chymotrypsin and trypsin, such as α-chymotrypsin and trypsin type 1, or combination thereof. According to one embodiment, the dosage form comprising the ionically chargeable hydrogel material is capable of reducing the activity of one or more proteases by at least 15%, at least 20%, at least 25%, at least 30%, at least 45%, at least 50% over time interval of 10 mins, 15 mins, 20 mins, 30 mins, 40 mins, 60 mins as determined by a Protease Activity Assay. According to a further embodiment, the dosage form comprises ionically chargeable hydrogel material that reduces the activity of one or more proteases, and includes an active agent that is a peptide. According to yet another embodiment, the active agent has GIP-1 agonist activity or GIP/GLP1 co-agonist activity. According to yet another embodiment, the active agent peptide is selected from any of semaglutide, exenatide, liraglutide, lixisenatide, albiglutide, dulaglutide, tirzepatide, and combinations thereof.

According to another embodiment, the dosage form comprising the delivery device with ionically chargeable hydrogel material, and methods therewith, maintains or increases the concentration of the active agent even in the presence of proteases at the intestinal site, relative to a delivery device that is absent the ionically chargeable hydrogel material. According to one embodiment, the dosage form comprising the ionically chargeable hydrogel material maintains or increases the concentration of the active agent in an Active Agent Concentration Assay in the presence of proteases over a time interval of at least 20 mins, at least 40 mins, or at least 60 mins. According to another embodiment, the concentration of the active agent provided by the delivery device having the ionically chargeable hydrogel material, as determined by an Active Agent Concentration Assay, is higher than the concentration of the active agent provided by a delivery device without ionically chargeable hydrogel material, over a time interval of at least 20 mins, at least 40 mins, or at least 60 mins. According to one embodiment, the concentration of the active agent is maintained or increased in the presence of proteases comprising chymotrypsin (e.g. α-chymotrypsin), trypsin (e.g. trypsin type 1), or a combination of both. According to one embodiment the dosage form and methods therewith comprises an active agent that is susceptible to degradation by a protease at the intestinal site. For example, the dosage form may be capable of inhibiting protease activity so as to maintain or increase levels of active agent that would otherwise be susceptible to degradation at the intestinal site.

The following examples are provided to further illustrate the methods of the present disclosure. These examples are illustrative only and are not intended to limit the scope of the disclosure in any way.

### EXAMPLES

Certain examples herein demonstrate results with the anionically chargeable peptides GG-353 and GG-427, which are described in US patent application publication no. 2020/0024322 (see e.g., Examples 2 and 4 and Table 13), hereby incorporated by reference herein in its entirety, and which act as GIP/GLPI co-agonists (see e.g. Tables 1 and 10). According to the published descriptions, GG-353 has a molecular weight of approximately 4928 g/mole and GG-427 has a molecular weight of approximately 4926 g/mole. Both structures include numerous carboxylic groups that are expected to be negatively charged at pH 5-8.

### Example 1

This example provides the recipe, manufacturing procedure and testing criteria of a representative particulate hydrogel according to the present disclosure. The representative hydrogel, named Hydrogel #3.02, contains the following ingredients:

| | Amount (g) |
|---|---|
| DI H₂O | 40 |
| AMPS¹ | 10 |
| BIS² | 0.2 |
| NaOH | 2.1 |
| 20% AP³ | 0.4 |
| 20% TEMED⁴ | 0.4 |
| SUM | 53.1 |

¹AMPS = 2-acrylamido-2-methylpropane sulfonic acid;
²BIS = methylenebisacrylamide 2% (crosslinker);
³AP = ammonium persulfate;
⁴TEMED = tetraethylenediamine

To obtain Hydrogel #3.02, the dry ingredients were added into fluid, and dissolved completely. Then ammonium persulfate was added and stirred well. Next TEMED was added and stirred, and the mixture was allowed to polymerize and cure overnight. The cured gel was broken into −1 cm chunks and added to 2 L of DI water under stirring. After 30 minutes of soaking, the pH of the mixture was measured. 1 N HCl was then added until the pH was adjusted to 7 (+/−0.2). The mixture was allowed to soak another 30 minutes under stirring. The fluid was drained and replaced with an additional 2 L of DI water. The conductivity of the fluid was measured to determine purity. The mixture was then allowed to soak under stirring for another 30 minutes. The fluid was drained and replaced with 400 mL isopropyl alcohol, and the mixture was allowed to dehydrate for 1 hour. The fluid was drained, and the gels were poured into a tray and allowed to dry in 158° F. oven overnight. The dried gels were ground into 500 μm-2000 μm particles.

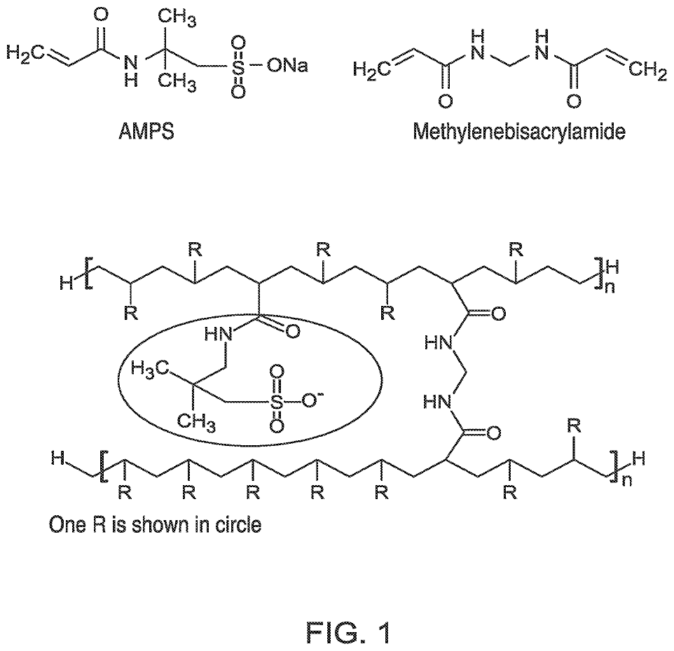

The standard Hydrogel #3.02 contains two monomers: 98% AMPS (2-acrylamido-2-methyl propane sulfonic acid) and 2% BIS (Methylenebisacrylamide) (as crosslinker) (FIG. 1). To understand the manufacturing consistency of Hydrogel #3.02 and how varying amounts of crosslinker may affect its key properties, tests that determine swell of the produced hydrogel and exclusion of sample agent were performed. Three samples (A, B and C) for each of the following hydrogel variations were produced for testing purpose: (1) using+20% crosslinker (0.24 g BIS); (2) using+10% crosslinker (0.22 g BIS); (3) using a standard amount of crosslinker (0.20 g BIS); (4) using −10% crosslinker (0.18 g BIS); and (5) using −20% crosslinker (0.16 g BIS).

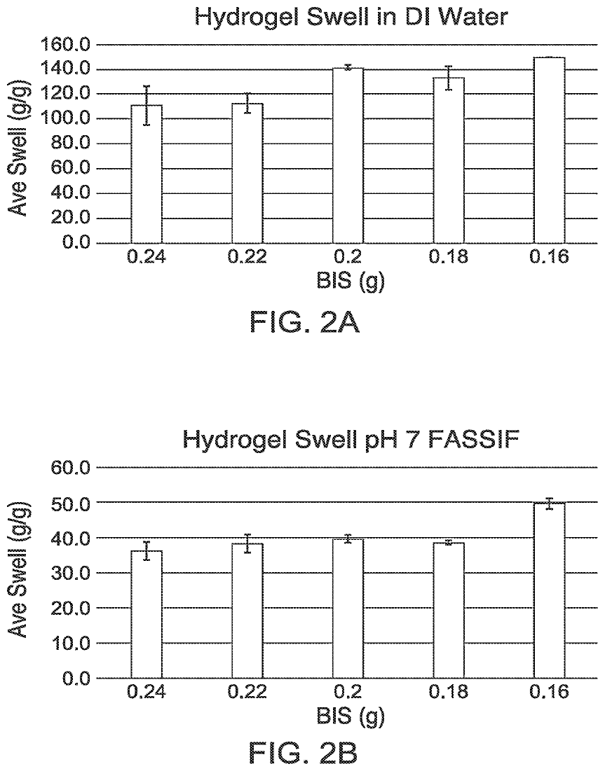

Swell of the hydrogels were conducted in both DI water and pH 7 FASSIF (Fasted State Simulated Intestinal Fluid) by: adding 0.1 g dried hydrogel to 15 mL fluid; soaking 15 minutes under gentle rocking; draining unabsorbed fluid via 500 μm mesh; and measuring unabsorbed fluid and calculating swell. The results were shown in the table below and in FIGS. 2A-2B.

| | | | | | |
|---|---|---|---|---|---|
| BIS (g) | A | B | C | AVG SWELL X (g/g) | STD |

SWELL, DI WATER

| | | | | | |
|---|---|---|---|---|---|
| 0.24 | 107 | 128 | 97 | 110.7 | 15.8 |
| 0.22 | 117 | 104 | 118 | 113.0 | 7.8 |
| 0.2 | 141 | 144 | 140 | 141.7 | 2.1 |
| 0.18 | 143 | 132 | 124 | 133.0 | 9.5 |
| 0.16 | 150 | 150 | 150 | 150.0 | 0.0 |

SWELL, pH 7 FASSIF

| | | | | | |
|---|---|---|---|---|---|
| 0.24 | 34 | 36 | 39 | 36.3 | 2.5 |
| 0.22 | 38 | 36 | 41 | 38.3 | 2.5 |
| 0.2 | 39 | 41 | 39 | 39.7 | 1.2 |
| 0.18 | 39 | 39 | 38 | 38.7 | 0.6 |
| 0.16 | 50 | 51 | 48 | 49.7 | 1.5 |

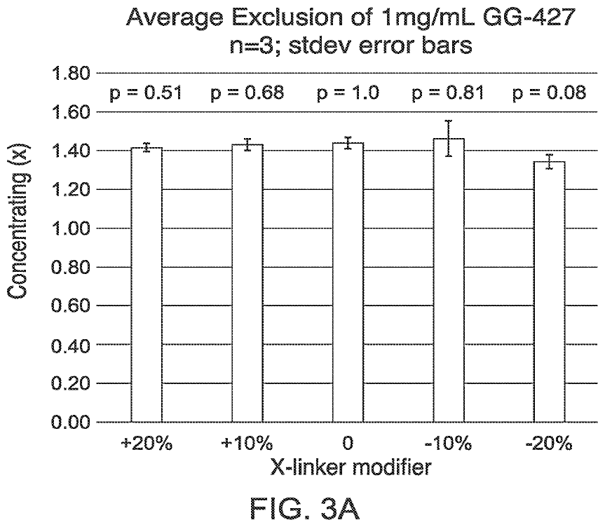

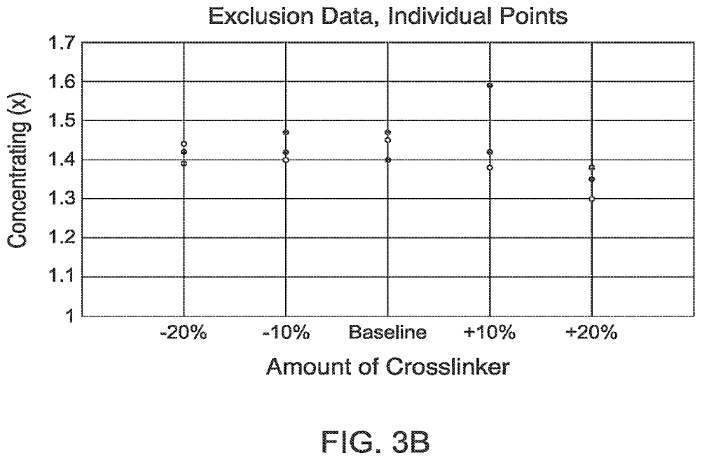

The exclusion test was carried out in pH 7 FASSIF with GG427 as the sample agent. The swell results in FASSIF from the table above was used to calculate amount of gel needed to absorb 8 mL FASSIF. The calculated amount of gel was then placed in 10 mL of 1 mg/mL GG427 in 15 mL tube under gentle rocking (active group). The same amount of gel was also placed in 10 mL of pH 7 FASSIF in 15 mL tube under gentle rocking (background group). Every 5 minutes for 25 minutes, 3×10 μL samples were extracted and diluted into 190 μL DI water in 96-well UV plate. Also, 3×10 μL samples of 1 mg/mL GG427 and pH 7 FASSIF stock solutions were taken, as controls. After completion, the absorbance at 230 nm and 265 nm was measured. The background was subtracted from active absorbance readings at each time point and compared to the stock solution to calculate the exclusion effect (Exclusion Amount). Results were shown in FIGS. 3A-3B. A consistent concentrating effect of 1.4× after 25 minutes was observed, indicating 80% fluid uptake into the hydrogel. That is, drug concentration in remaining free 20% of fluid is 1.4× higher than the starting concentration.

### Example 2A

The present example illustrates in vivo protocols for particulate hydrogels disclosed herein.

### Oral Capsule Dosing

Beagle dogs (same animals as for subcutaneous dosing, n=4 or 8) were fasted overnight prior to dose administration (˜12 hours). To stimulate gastric secretion, each animal received a single 6 μg/kg intramuscular injection of pentagastrin approximately 30 minutes prior to dose administration. Food was returned following the 6-hour sample collection. Oral administration of capsules was followed by a 10 ml flush of saline. Blood samples were collected Pre-dose, 0.25, 0.5, 1, 2, 3, 4, 6, 8, and 24 hours post-dose. Blood samples were be collected via cephalic venipuncture into tubes (1.3 mL K3EDTA) containing 3 μL of commercial aprotinin solution and 1.3 μL of commercial dipeptidyl peptidase 4 (DPP IV) inhibitor. The blood tubes were vortex mixed once the whole blood sample was added. Sample tubes were be stored on wet ice until processed to plasma by centrifugation (3500 rpm at 5° C. for 10 minutes) within 30 minutes of collection. Plasma samples were transferred into matrix tubes and stored at nominal −70° C. until shipment to a contract laboratory for analysis of the proprietary peptides present.

Capsules were size 00 or 000 gelatin capsules with enteric coating (Eudragit S100) designed to open in the small intestine. The drug formulation consisted of 5-24 mg of peptide drug, 200-500 mg of sodium caprate, zero or 125 mg of soybean trypsin inhibitor (GG-353 used 125 mg, GG-427 used zero mg), and zero or 300 mg of hydrogel.

### Subcutaneous Dosing to Permit Oral Bioavailability Calculations

Beagle dogs (same animals as for capsule dosing; n=4 or 8) were fasted overnight prior to dose administration (˜12 hours). Food was returned following the 6 hour sample collection. A subcutaneous dosing solution was made to 0.5 mg/mL using the proprietary peptide being studied in 40 mM Tris buffer at pH 8. Dose formulations were prepared by adding pre-weighed drug to the appropriate amount of vehicle to result in the designated dose concentration. The vehicle used forformulations was pre-filtered prior to use. All dose syringes were weighed prior to and following dosing to gravimetrically determine the amount of formulation administered, delivering ˜0.02 mL/kg for a final drug dose of 0.01 mg/kg.

Blood samples were collected Pre-dose, 0.25, 0.5, 1, 2, 3, 4, 6, 8, and 24 hours post-dose. Blood samples were be collected via cephalic venipuncture into tubes (1.3 mL K3EDTA) containing 3 μL of commercial aprotinin solution and 1.3 μL of commercial dipeptidyl peptidase 4 (DPP IV) inhibitor. The blood tubes were vortex mixed once the whole blood sample was added. Sample tubes were be stored on wet ice until processed to plasma by centrifugation (3500 rpm at 5° C. for 10 minutes) within 30 minutes of collection. Plasma samples were transferred into matrix tubes and stored at nominal −70° C. until shipment to a contract laboratory for analysis of the proprietary peptides present.

### Bioavailability Calculations

Oral bioavailability was calculated by determining the drug concentration area under the curve (AUC) between dosing and 24 hours for both the oral and subcutaneous dosing. The percent bioavailability was calculated by determining the ratio of the oral AUC to the subcutaneous AUC, corrected for the mg/kg of drug dosed to each animal by each route of administration, and multiplied by 100%.

### Results

### Hydrogel 1

Experiment 1. Oral bioavailability in dogs (n=8 in each arm)
Peptide 1 without hydrogel 2.9% bioavailability vs subcutaneous, CV (coefficient of variation)=1.37.
Peptide 1 with 300 mg hydrogel 10.8% bioavailability vs subcutaneous, CV (coefficient of variation)=1.32.
Experiment 2. Oral bioavailability in dogs (n=8 in each arm), with pentagastrin to acidify stomach.
Peptide 1 without hydrogel 2.5% bioavailability vs subcutaneous, CV (coefficient of variation)=1.03.
Peptide 1 with 300 mg hydrogel 6.3% bioavailability vs subcutaneous, CV (coefficient of variation)=0.79.
Experiment 3. Oral bioavailability in dogs, with pentagastrin to acidify stomach.
Peptide 2 without hydrogel (n=7) 2.6% bioavailability vs subcutaneous, CV (coefficient of variation)=0.75.
Peptide 2 with 300 mg hydrogel (n=4) 6.6% bioavailability vs subcutaneous, CV (coefficient of variation)=0.43
   Hydrogel 2 (Lower Crosslinking Amount than Hydrogel 1)
Experiment 3. Oral bioavailability in dogs, with pentagastrin to acidify stomach
Peptide 2 without hydrogel (n=7) 2.6% bioavailability vs subcutaneous, CV (coefficient of variation)=0.75
Peptide 2 with 300 mg hydrogel (n=4) 5.4% bioavailability vs subcutaneous, CV (coefficient of variation)=1.04.

Studies were also performed with GG-353 (referred to as “Peptide A”), GG-427 (referred to as “Peptide B”) and a large molecule hormone analog that is ionically chargeable at pH of 4 to 8 (referred to as “Peptide C), which comprises multiple chargeable carboxyl groups and a molecular weight between 4000-5000 g/mol. These studies used #3.02 hydrogel particulates sieved through a 63-micron sleeve, and bioavailability was determined as per the procedure described above. For “Study 1” with GG-353 (“Peptide A”), the dose of GG-353 was split evenly over 5 capsules. For “Study 2” with GG-353 (“Peptide A”), the entire dose was provided in a single larger capsule. For “Study 1” and “Study 2” with GG-427 (“Peptide B”), bioavailability with (“Study 2”) and without (“Study 1”) pentagastrin dosing to animals was compared. Pentagastrin dosing was performed to ensure the stomach pH of the dosed animals was similar to that of humans (pentagastrin lowers gastric pH). Pentagastrin dosing was performed by intramuscular injection of 5 mg of pentagastrin 30 minutes before capsule dosing.

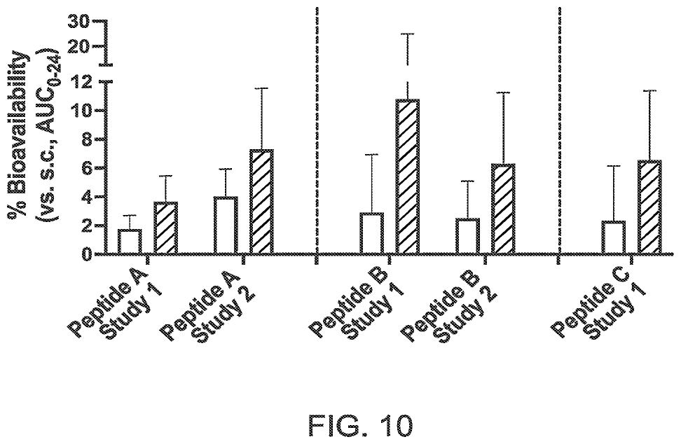

The results for the bioavailability for each of the peptides is shown in FIG. 10, for samples containing the hydrogel as well as samples that did not contain any hydrogel. The results show that those samples having hydrogel significantly and markedly increased the uptake and bioavailability of the peptides.

### Example 2B

In this example, the in vivo evaluation of a representative dosage form as described herein was provided. The representative dosage form comprises: ordinary enteric capsule or tablet (size 0), GG-353 (active agent), sodium caprate (permeation enhancer), soybean trypsin inhibitor and Hydrogel #3.02 particles (˜1 mm³ each). The mechanism of action is as follows: capsule dissolves in small intestine; hydrogel selectively absorbs water, leaving behind a higher concentration of other ingredients; higher concentration of permeation enhancer opens tight junctions more; higher concentration of SBTI inhibits pancreatic enzymes more; and higher drug concentrations drives faster uptake through tight junctions.

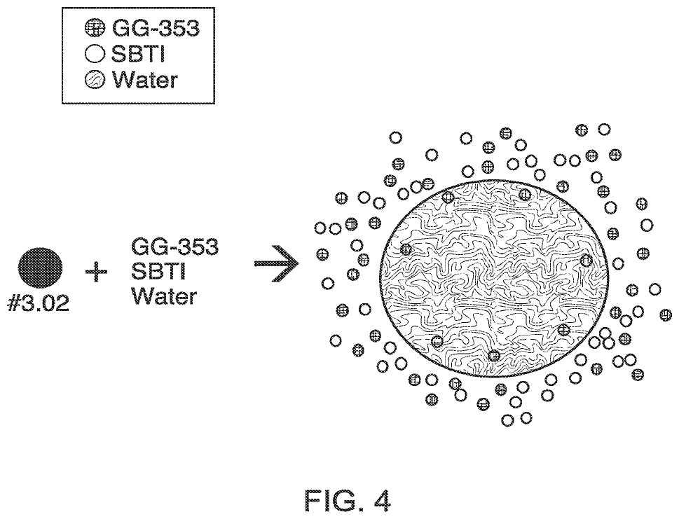

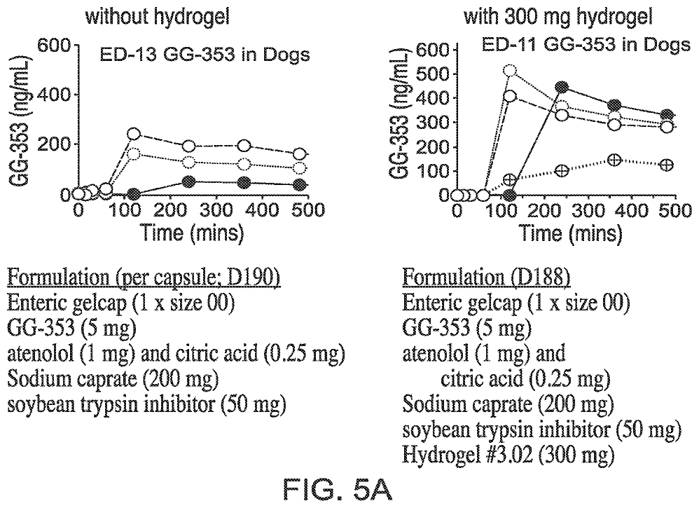

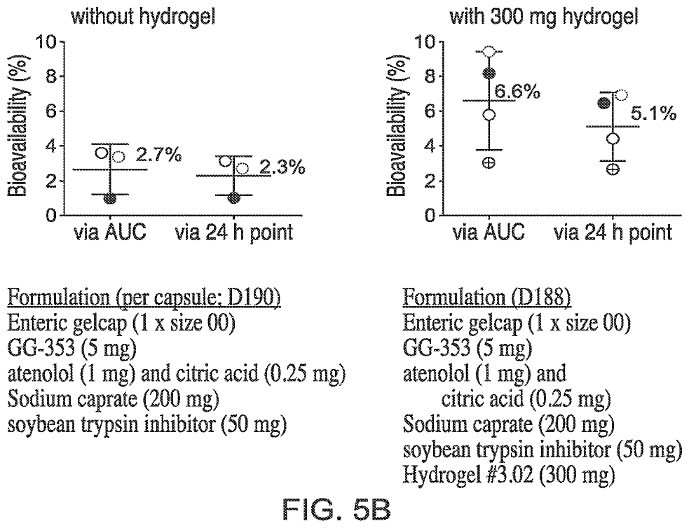

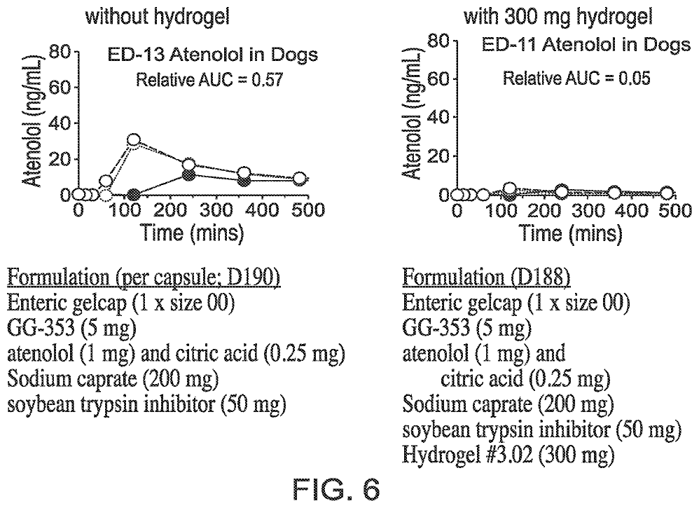

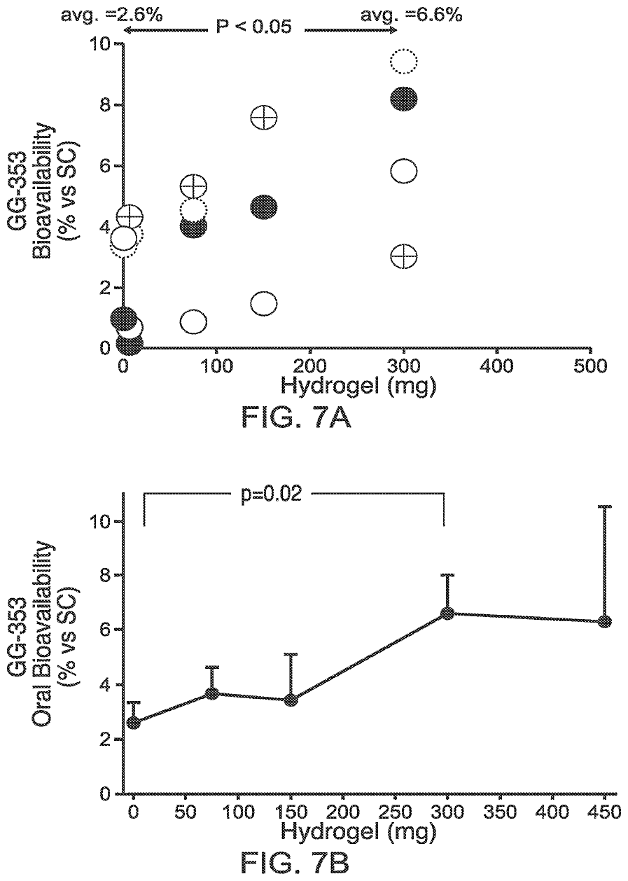

Hydrogel #3.02 is nonporous, and has high anionic charge (via attached sulfonate groups). It is lightly crosslinked, which leads to high swelling in aqueous solution. GG-353 has many negatively charged carboxylate groups. Caprate and caprate micelles are also negatively charged. As shown in FIG. 4, high negative charge of Hydrogel #3.02 deters entry of negatively charged molecules (known as the Donnan equilibrium). Meanwhile, lack of channels through the gel leads to exclusion by size: SBTI (181 amino acids/20 kDa) is too large to penetrate the gel, and peptides penetrate the swelling hydrogel by diffusion far more slowly than water, which leads to excess water in interior relative to near surface regions. The in vivo results were shown in FIGS. 5A-5B and 6. Comparing oral bioavailability to hydrogel content throughout all samples, it indicates that 300 mg hydrogel yields 2.5× bioavailability compared to no hydrogel as shown in FIG. 7A (FIG. 7B provides another visualization of the same data in the form of a line graph with error bars, and including a data point for 450 mg hydrogel).

### Example 2C

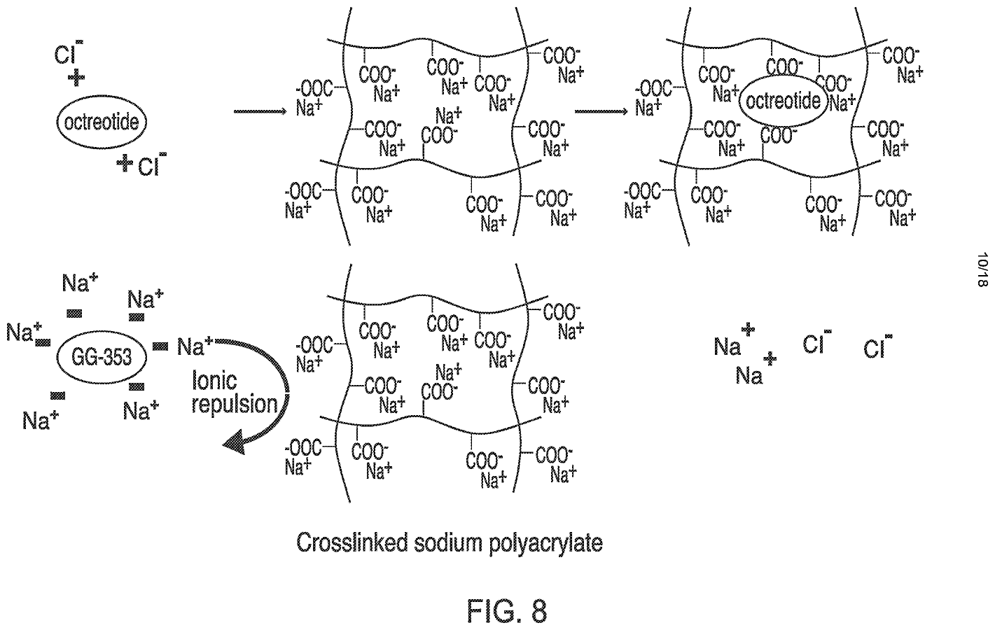

The present example illustrates the results of some representative agents having different ionic charges. Using Hydrogel #3.02 as an example, because this hydrogel has a net anionic charge, drugs having a net anionic charge can be effectively excluded from the swelling hydrogel, while those having a net cationic charge are likely to be trapped inside (FIG. 8).

### Anionic Agents

### Exenatide

Exenatide (exenadin acetate) is an FDA approved therapeutic peptide with a molecular weight of 4,187 g/mole and a net negative charge at pH above 5 (isoelectric point of exenatide=4.86). Exenatide (5 mg) was dissolved in 5 mL of modified FASSIF buffer at pH 7. Hydrogel #3.02 (300 mg) was added, absorbing approximately 80% of the fluid over 25 minutes. Over multiple repetitions, the concentration of exenatide in the exterior fluid was determined by UV absorbance to be 1.75-2 mg/mL after 25 minutes, demonstrating partial exclusion of the peptide from the hydrogel.

### Sodium Caprate

Sodium caprate (sodium decanoate) is a known intestinal permeation enhancer with a negative charge (negative charge on the caprate). Sodium caprate (280 mg) was dissolved in 5 mL of modified pH 7 FASSIF buffer. Hydrogel #3.02 (300 mg) was added, absorbing approximately 80% of the fluid. Over multiple repetitions, the concentration of caprate in the exterior fluid was determined by UV absorbance to be ˜1.4 times the initial concentration before hydrogel was added, demonstrating partial exclusion of the caprate from the hydrogel.

### Exenatide Plus Sodium Caprate

Similar to the experiments above, exenatide (5 mg) was dissolved in 5 mL of modified FASSIF buffer at pH 7. The intestinal permeation enhancer, sodium caprate (280 mg) was added. Hydrogel #3.02 (300 mg) was added, absorbing approximately 80% of the fluid. Over multiple repetitions, the concentration of exenatide in the exterior fluid was determined by UV absorbance to be 1.6 mg/mL, demonstrating partial exclusion of the peptide from the hydrogel in the presence of sodium caprate.

### Cationic Agents

### Octreotide

Octreotide (as octreotide acetate) is an FDA approved therapeutic peptide with a molecular weight of 1019 g/mole and a net positive charge at pH below 8 (isoelectric point of octreotide=8.5). Octreotide (5 mg) was dissolved in 5 mL of modified FASSIF buffer at pH 7 (resulting in a starting concentration of 1 mg/mL). Hydrogel #3.02 (300 mg) was added, absorbing approximately 80% of the fluid. Over multiple repetitions, the concentration of octreotide in the exterior fluid was determined by UV absorbance to be less than 0.8 mg/mL after 10 minutes, demonstrating binding (rather than exclusion) of the peptide from the hydrogel.

### Lanreotide (in FASSIF)

Lanreotide is an FDA approved therapeutic peptide with a molecular weight of 1096 g/mole and a net positive charge at pH below 8. Lanreotide (5 mg) was dissolved in 5 mL of modified FASSIF buffer at pH 7. Hydrogel #3.02 (300 mg) was added, absorbing approximately 80% of the fluid over 25 minutes. Over multiple repetitions, the concentration of lanreotide in the exterior fluid was determined by UV absorbance to be ˜0.3 mg/mL after 25 minutes, demonstrating binding (rather than exclusion) of the peptide from the hydrogel.

### Lanreotide (in DI Water)

Lanreotide is an FDA approved therapeutic peptide with a molecular weight of 1096 g/mole and a net positive charge at pH below 8. Lanreotide (5 mg) was dissolved in 5 mL of deionized water. Hydrogel #3.02 (300 mg) was added, absorbing approximately 80% of the fluid over 25 minutes. Over multiple repetitions, the concentration of lanreotide in the exterior fluid was determined by UV absorbance to be less than 0.05 mg/mL after 25 minutes, demonstrating binding (rather than exclusion) of the peptide from the hydrogel.

### Example 3

This example provides data on the level of a peptide present outside the hydrogel when combined with protease and the hydrogel in a fluid such as water, as well as the measured activity of proteases (chymotrypsin, trypsin) when combined with hydrogel.

A Protease Activity Assay was used to determine levels of proteases. Protease activity for proteases comprising chymotrypsin or trypsin were determined, by using either alpha-chymotrypsin, CAS: 9004-07-3 or Trypsin Type 1, CAS: 9002:07-7. A chymotrypsin activity assay kit was used, available from Abcam, including coumarin substrate, chymotrypsinogen activator, and a chymotrypsin assay buffer (“cTAB”). A trypsin activity assay kit was also used, available from Abcam, including p-NA substrate, and Trypsin assay buffer (“TAB”). Materials for the assay further comprised Tris solution having a pH of 7.4 with 0.5 mM Ca.

To prepare the Protease Activity Assay for chymotrypsin, the following steps were performed: the hydrogel was massed (hydrogel #3.02 described in Example 1) in 75 mg aliquots into a labeled 15 mL tube; an additional 15 mL tube was labelled as the control; aliquots of the reaction solution were created (per Abcam assay kit instructions) by combining 46 microliters of cTAB, 2 microliters of Coumarin substrate, and 2 microliters of chymotrypsinogen activator in each aliquot (e.g. to provide for 4 timepoints, n=3 all samples, 2 tubes=24 aliquots total required, so total solution was 1104 microliters of cTAB, 48 microliters of Coumarin substrate, and 48 microliters of chymotrypsinogen activator); the reaction solution was vortexed to mix; the sample background control (SBC) solution was created (e.g. per Abcam assay kit instructions) by combining, for one aliquot, 48 microliters of cTab and 2 microliters of chymotrypsinogen activator (e.g. to provide for 4 timepoints=4 aliquots required, so total solution was 192 microliters of cTab and 8 microliters of chymotrypsinogen activator); the solution was vortexed to mix; and 10 μg/mL chymotrypsin solution in tris buffer was created (e.g., 15 mL total solution for test with 5 mL per tube). The procedure for the chymotrypsin activity assay included adding 5 mL of chymotrypsin solution in tris buffer to each of the sample tubes containing dry hydrogel, and empty control tubes, beginning the timer and mixing with a spatula to break up any hydrogel as needed. The tubes were then incubated at 150 RPM and 39° C. Sampling from each of the tubes was performed at each time point (5, 15, 30 and 60 minutes) by taking 10 microliters from each tube, and combining into 50 microliters of the reaction solution described above, with n=3 tubes, for a total of 6 samples per time point. The samples were provided in wells of a UV-clear, flat-bottom plate (e.g. 96-well plate), and 50 microliters of the SBC solution above were provided in a single well as a moving background. The fluorescent readings were read at 360/460 nm for the respective sample wells at the time points. For each tube at each time point and incubation, the three readings for a single output were averaged, and the SBC solution reading was subtracted from each of the averages. For each average, calculation of the activity relative to the initial control reading was made for each incubation.

To prepare the Protease Activity Assay for trypsin, the following steps were performed: the hydrogel was massed (hydrogel #3.02 described in Example 1) in 75 mg aliquots into labeled 15 mL tube; an additional 15 mL tube was labelled as the control; aliquots of the reaction solution were created (per the Abcam assay kit) by combining 48 microliters of TAB and 2 microliters of p-NA substrate in each aliquot (e.g. to provide for 4 time points, n=2 all samples, 3 tubes=26 aliquots total required, so total solution was 1248 microliters of TAB and 52 microliters of p-NA substrate); the reaction solution was vortexed to mix; and a 150 microgram per liter trypsin solution in tris was created (e.g. per Abcam assay kit instructions) with 5 mL per tube for a total of 15 mL. The procedure for the trypsin activity assay included adding 5 mL of trypsin solution in tris buffer to each of the sample tubes containing dry hydrogel, and empty control tubes, and beginning the timer and mixing with a spatula to break up any hydrogel as needed. The tubes were then incubated at 150 RPM and 39° C. Two 50 microliter reaction solution aliquots were placed in a separate well of the plate as a background. Sampling from each of the tubes was performed at each time point (5, 15, 30, 60 minutes). Each of the 50 microliter samples were placed in 450 microliters of TAB and vortexed to mix. Then from each solution 10 microliters was taken from each tube, and combined into 50 microliters of the reaction solution described above. The samples were provided in wells of a UV-clear, flat-bottom plate (e.g. 96 well plate). The fluorescent readings were read at 405 nm for the respective sample wells at the timepoints. For each tube at each timepoint and incubation, the average of 2 readings for a single output were averaged, and the average of the reaction solution background readings was subtracted from all averages. For each average after background subtraction, calculation of the activity relative to the initial control reading after background subtraction was made for each incubation.

To determine levels of active agent present in bulk fluid when combined with hydrogel, 30 mg/mL of the peptide C (a large molecule hormone analogue with multiple CO2H groups and a molecular weight between 4000-5000 g/mol, and which is ionically chargeable at pH of 4 to 8) was combined with 1 mg/mL ionically chargeable hydrogel (hydrogel #3.02 described in Example 1) and protease (chymotrypsin ortrypsin) in a buffered fluid. The amount of Peptide C in the fluid (i.e. outside the hydrogel) was determined at an initial data point, and at 30 minutes and 60 minutes after the initial data point, by a liquid chromatography-mass spectrometry technique.

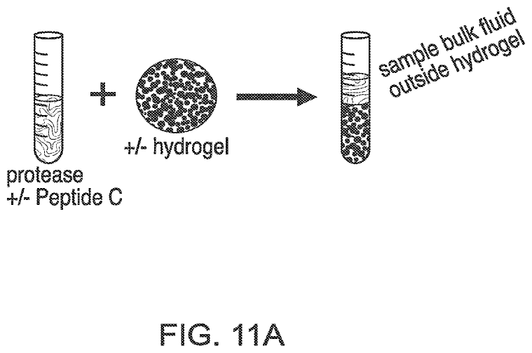

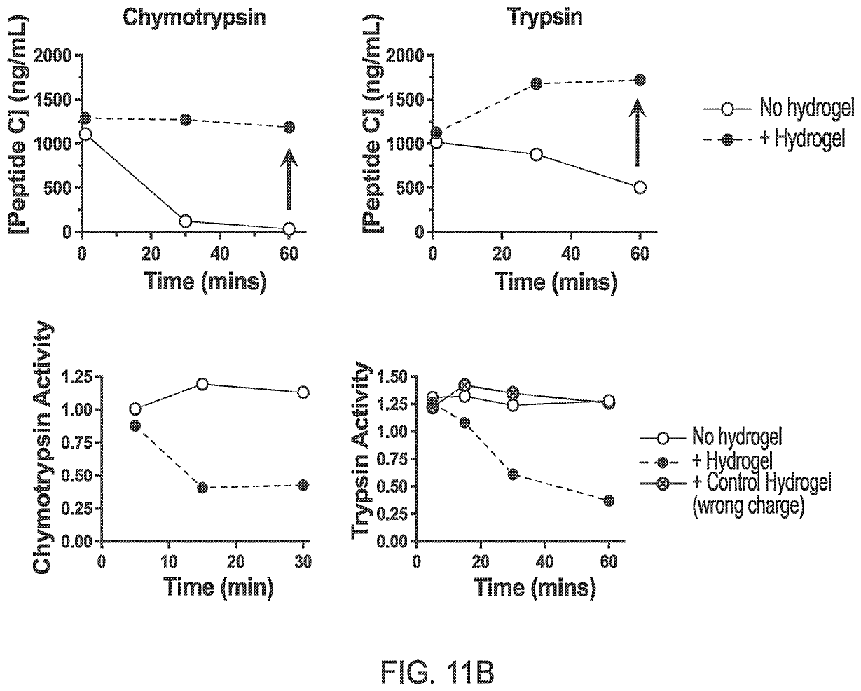

As is depicted in FIGS. 11A-11B, in vitro assays have shown that for a combination of the hydrogel with the peptide C, the combination of hydrogel with peptide C, where the hydrogel have a net ionic charge having a sign that is the same as the sign of the net ionic charge of the peptide C, maintains or even increases the concentration of the peptide overtime, despite being in the presence of the protease which would tend to degrade the peptide C (see upper graphs in FIG. 11B, right hand for trypsin, left hand for chymotrypsin). This is as compared to solution containing peptide C and protease, but without hydrogel, which exhibits a marked decrease in peptide levels over time.

In addition, combination of peptide C with the hydrogel leads to a reduction in overall protease activity (chymotrypsin and trypsin) as is depicted in FIG. 11B. Activity levels of trypsin and chymotrypsin were relatively stable and even increased over time without hydrogel, but the activity significant decreased in the presence of hydrogel (see lower graphs in FIG. 11B, right hand for trypsin, left hand for chymotrypsin). Also, testing of an ionically chargeable hydrogel having a same overall sign of its net charge to that of trypsin, showed that this hydrogel had little effect on trypsin activity, demonstrating that the effect in reducing protease activity may be at least partly due to the opposite-charge effect of ionically chargeable hydrogel having an opposite sign of its net ionic charge as the net ionic charge of the protease (see lower graphs in FIG. 11B, right hand for trypsin, notated as “wrong charge”).

### Example 4A

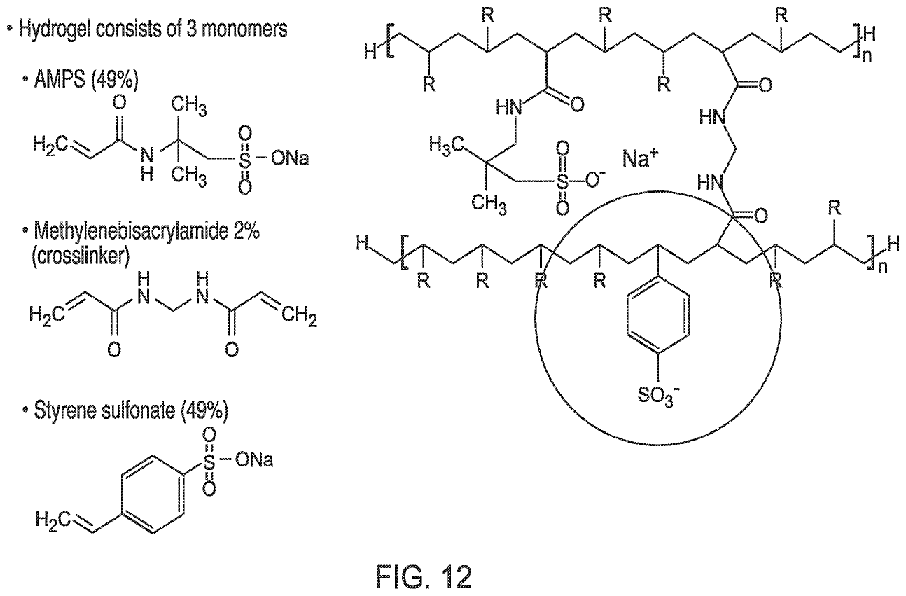

This example provides the recipe, manufacturing procedure and testing criteria of another representative particulate hydrogel, named Hydrogel #12.035, according to the present disclosure. As shown in FIG. 12, Hydrogel #12.035 is a crosslinked copolymer with 2 monomers (AMPS and styrene sulfonate) and a crosslinker (methylenebisacrylamide). The circled part in FIG. 12 highlights the difference from hydrogel 3.02. Without being limited to any particular theory, the addition of styrene sulfonate (also called vinylbenzene sulfonate) was provided in the interests of optimizing the chymotrypsin binding to the hydrogel by providing hydrophobic regions on the benzene ring to interact with hydrophobic portions of the chymotrypsin enzyme, thereby reducing interactions of the active agent with the chymotrypsin enzyme to inhibit the degradation thereof.

### Hydrogel 12.035 Manufacturing

In a 200 mL glass beaker, the following materials were added to 40 g deionized water (DI) under stirring at room temperature: 5 g 2-Acrylamido-2-methylpropane sulfonic acid (AMPS), 5 g 4-vinylbenzenesulfonate (4-VBS), 1 g Sodium Hydroxide (NaOH), and 0.35 g Methylenebis(acrylamide) (BIS).

The solution was stirred until clear and materials were fully dissolved. While materials were stirring, initiator solutions were created containing: Ammonium Persulfate (20% Ammonium Persulfate in 80% DI by mass), and Tetramethylethylenediamine (TEMED) (20% TEMED in 80% DI by mass).

Once all solutions were clear and fully dissolved, a Nitrogen (N₂) line was placed into the hydrogel solution and N₂ gas was bubbled into the 200 mL hydrogel solution for two minutes. After two minutes, with the N₂ line still in place, 400 μL of Ammonium Persulfate solution was added into beaker immediately followed by 400 μL of TEMED solution. The N₂ line was removed and the beaker was covered. The solution was allowed to polymerize and cure overnight.

Following overnight curing, the hydrogel was removed from beaker and broken into sub-1 cm chunks. The chunks were placed in a 2 L beaker and 1800 mL of DI water was under stirring at room temperature. After 15 mins, the pH of the mixture was measured. 1 N HCl was added in small increments until the mixture pH was approximately 7.0 (+/−˜0.1). Around 1.0-1.5 mL of HCl is expected to be required. After final HCl was added, the mixture was allowed to stir an additional 30 minutes. After 30 minutes, excess DI water was drained from swollen gel and replaced with 1800 mL fresh DI water. The mixture was allowed to stir 30 minutes then drained and replaced fluid. This process was repeated until a total of six washes were completed. After the final DI water wash, all excess water was drained and 500 mL of 100% Isopropanol (IPA) was poured into the beaker with gel. The mixture as allowed to stir 30 minutes. After 30 minutes, the IPA was drained, the hydrated gel chunks were placed in a tray, and the tray was placed in a 160F oven overnight or until fully dry. Once gel was completely dry, gel particles were ground using a particle mill or coffee grinder. Using a 63 m sieve, particles were repeated sifted, and particles larger than 63 m were re-ground.

### Evans Blue Exclusion Procedure

Commercially obtained Evan's blue dye was used as a surrogate marker for an anionic drug in hydrogel testing. It has a molecular weight of 961.8 g/mole and has four negative charges at pH 7 due to four sulfonate groups in the structure. Its visible light absorbance at 605 nm provides an easy way to monitor the concentration of the dye when exposed to the hydrogel in solution.

FASSIF buffer was created by adding 41.65 g of FASSIF buffer concentrate (Biorelevant Media, code FaSSIF) to 961.1 g DI water and stirred until completely mixed. Evans Blue solution was created by adding Evans Blue to approximately half of the FASSIF solution such that the final concentration of the media is 0.125 mg/mL Evans Blue.

5 mL of Evans Blue solution was added to a 15 mL centrifuge tube (EB Tube). 5 mL of FASSIF buffer (without Evans Blue) was added to another 15 mL tube (Background Tube). 200 mg of sub-63 um hydrogel #12.035 was added to both tubes. Vortexed tubes until fully mixed and placed both tubes on rocker plate for 15 minutes. After 15 minutes, both tubes were centrifuged at 3000 g for 1 minute. (N=3) 20 μL samples were extracted from the supernatant of both tubes and diluted into 180 μL of DI water in 96-well UV-clear plate. (N=3) samples (20 μL) of 0.125 mg/mL of Evans Blue solution (EB Stock) and FASSIF buffer (FASSIF Stock) were also added to 180 μL DI water in same plate. The plate was read for absorbance at 605 nm.

The absorbance reading samples from each group were averaged. The following formula was used to calculate concentrating factor:

$$Concentrating X = \frac{EB Tube - Background Tube}{EB Stock - FASSIF Stock}$$

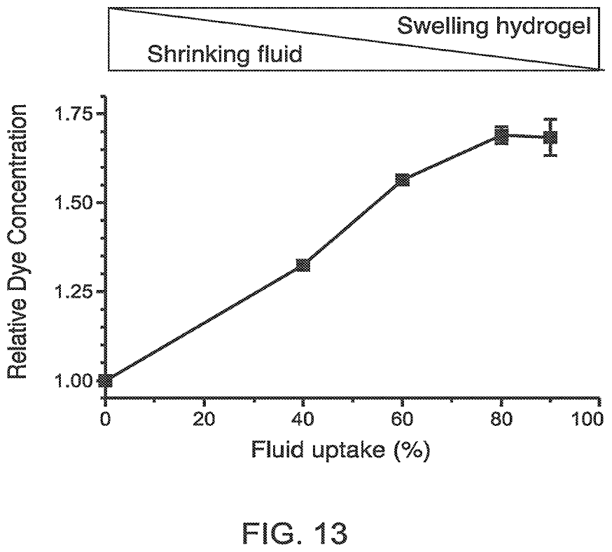

Results were provided in FIG. 13 showing the relative concentration of the dye when hydrogel #12.035 was added in varying amounts sufficient to absorb specific fractions of the fluid present in the FASSIF buffer at pH 7. For example, 200 mg absorbed approximately 80% of the fluid present in the 5 ml test. FIG. 13 clearly shows the increasing concentration of dye as more hydrogel was added. Each of the data points (40, 60, 80 and 90% fluid uptake) were tested multiple times and were statistically significant relative to the no hydrogel control (0% fluid uptake) with p<0.001 using a two tailed student t-test. Error bars shown are standard error of the mean.

### Example 4B

The present example illustrates in vivo protocols for Hydrogel #12.035, and using substantially the same procedure as described in Example 2A above.

### Oral Capsule Dosing

Beagle dogs (same animals as for subcutaneous dosing, n=8) were fasted overnight prior to dose administration (˜12 hours). To stimulate gastric secretion, each animal received a single 6 μg/kg intramuscular injection of pentagastrin approximately 30 minutes prior to dose administration. Food was returned following the 6-hour sample collection. Oral administration of capsules was followed by a 10 ml flush of saline. Blood samples were collected Pre-dose, 0.25, 0.5, 1, 2, 3, 4, 6, 8, and 24 hours post-dose. Blood samples were be collected via cephalic venipuncture into tubes (1.3 mL K3EDTA) containing 3 μL of commercial aprotinin solution and 1.3 μL of commercial dipeptidyl peptidase 4 (DPP IV) inhibitor. The blood tubes were vortex mixed once the whole blood sample was added. Sample tubes were be stored on wet ice until processed to plasma by centrifugation (3500 rpm at 5° C. for 10 minutes) within 30 minutes of collection. Plasma samples were transferred into matrix tubes and stored at nominal −70° C. until shipment to a contract laboratory for analysis of the proprietary peptides present.

Capsules were size 00 gelatin capsules with enteric coating (00EL) designed to open in the small intestine. The drug formulation consisted of 20 mg of Peptide C, 280 mg of sodium caprate, and zero or 300 mg of Hydrogel #12.035.

### Subcutaneous Dosing to Permit Oral Bioavailability Calculations

Beagle dogs (same animals as for capsule dosing; n=8) were fasted overnight prior to dose administration (˜12 hours). Food was returned following the 6 hour sample collection. A subcutaneous dosing solution was made to 0.5 mg/mL using the proprietary peptide being studied in 40 mM Tris buffer at pH 8. Dose formulations were prepared by adding pre-weighed drug to the appropriate amount of vehicle to result in the designated dose concentration. The vehicle used forformulations was pre-filtered prior to use. All dose syringes were weighed prior to and following dosing to gravimetrically determine the amount of formulation administered, delivering ˜0.02 mL/kg for a final drug dose of 0.01 mg/kg.

Blood samples were collected Pre-dose, 0.25, 0.5, 1, 2, 3, 4, 6, 8, and 24 hours post-dose. Blood samples were be collected via cephalic venipuncture into tubes (1.3 mL K3EDTA) containing 3 μL of commercial aprotinin solution and 1.3 μL of commercial dipeptidyl peptidase 4 (DPP IV) inhibitor. The blood tubes were vortex mixed once the whole blood sample was added. Sample tubes were be stored on wet ice until processed to plasma by centrifugation (3500 rpm at 5° C. for 10 minutes) within 30 minutes of collection. Plasma samples were transferred into matrix tubes and stored at nominal −70° C. until shipment to a contract laboratory for analysis of the proprietary peptides present.

### Bioavailability Calculations

Oral bioavailability was calculated by determining the drug concentration area under the curve (AUC) between dosing and 24 hours for both the oral and subcutaneous dosing. The percent bioavailability was calculated by determining the ratio of the oral AUC to the subcutaneous AUC, corrected for the mg/kg of drug dosed to each animal by each route of administration, and multiplied by 100%.

### Results

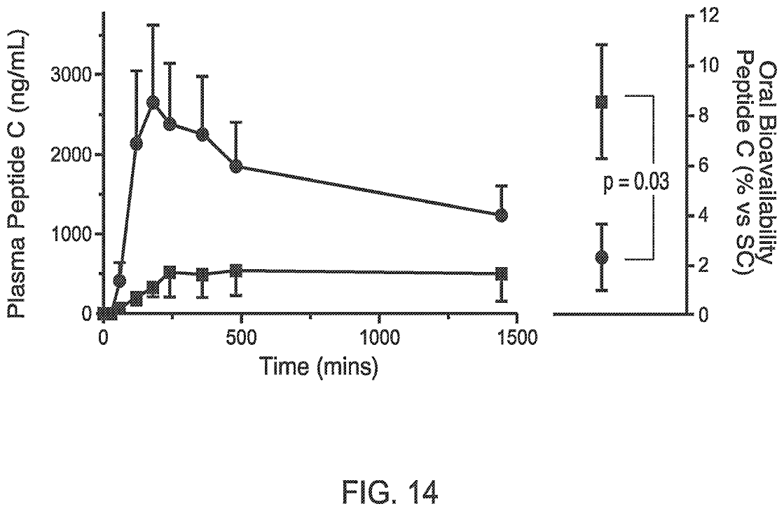

As shown in FIG. 14, the oral bioavailability of this hydrogel formulation was better than the control with p=0.03 in a two tailed student t-test. Error bars shown are standard error of the mean.

### Example 5

This example provides a direct comparison between the two hydrogels disclosed herein: Hydrogel #12.035 and Hydrogel #3.02. The experimental protocol for this in vitro test is summarized below:

Summary of Concentrations in Test Fluid with 5 mL Total Volume
Peptide C 1 mg/mL
α-Chymotrypsin type ∥ lyophilized powder from bovine pancreas (Sigma) 10 μg/mL
Hydrogel #12.035 or 3.02, 40 mg/mL (takes up ˜80% of fluid)

### Incubation Composition

200 mg Hydrogel (0 mg hydrogel in control)
1.67 mL buffer
1.67 mL drug stock
1.67 mL chymotrypsin stock

### Stock Solutions

Buffer: Tris-Ca tris-buffered saline+0.5 mM calcium chloride
Peptide C 1 mg/mL in Tris-Ca
Chymotrypsin 30 μg/mL in Tris-Ca
Stop solution 0.1 mg/mL soybean trypsin inhibitor (Sigma) tris-Ca

### Detailed Method

Each solution was preheated to 45° C. (the chymotrypsin solution was kept at room temperature) and placed in a separate 15 mL centrifuge tube.

Solutions were mixed manually with a spatula to break up hydrogel and vortexed.

Tubes were placed in the incubator at 150 RPM and 45° C. 45° C. was selected due to the large fraction of time outside the incubator for sampling, which keeps the temperature of the samples fairly close to 37° C.

### Sampling Procedure at Each Time Point:

Tubes were centrifuge pulsed up to 3000G;
Each tube had a 20 μL sample withdrawn and diluted in 480 μL stop solution;
Each diluted sample had a 20 μL sample diluted in a further 480 μL stop solution;
After each time point, the first sample tube was discarded and the second sample tube (from the second dilution step) was placed in the −80° C. freezer;
Tubes were mixed manually with a spatula and vortexed before being placed back in the incubator;

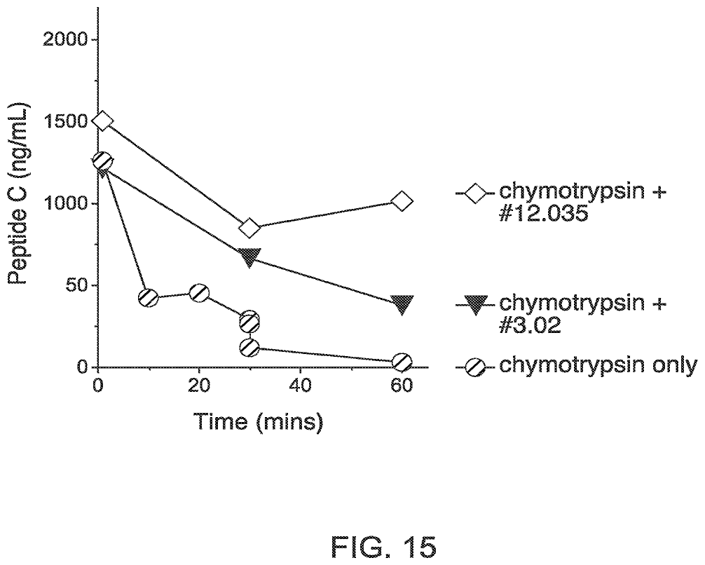

Analytical determination of Peptide C in thawed samples was performed by HPLC-MS. Values shown for Peptide C represent the concentration detected in the final stop solution sample (FIG. 15). The in vitro data indicates a better therapeutic potential for Hydrogel #12.035 compared to Hydrogel #3.02.

The following Enumerated Embodiments are provided to illustrate aspects of the disclosure, although the embodiments are not intended to be limiting and other aspects and/or embodiments may also be provided.

Embodiment 1: A pharmaceutically acceptable oral dosage form for delivery of an active agent to an intestinal site, the dosage form comprising:
a delivery device comprising:
   a plurality of hydrogel particulates comprising ionically chargeable hydrogel material, the ionically chargeable hydrogel material comprising a crosslinked polymer material having a Swelling Ratio of at least 5 in deionized water; and
   an ionically chargeable active agent, the ionically chargeable active agent having a net ionic charge with a sign that is the same as a sign of a net ionic charge of the ionically chargeable hydrogel at an intestinal pH in a range of from about 4 to about 8; and
a protective coating covering the delivery device.

Embodiment 2: The dosage form according to Embodiment 1, wherein the crosslinked polymer material has a Swelling Ratio of at least 10, at least 20, at least 25, at least 30, at least 40, at least 50, at least 60, at least 75, at least 85, at least 100, at least 110, at least 120, and/or 200 or higher, as measured in deionized water.

Embodiment 3: The dosage form according to any preceding Embodiment, wherein the crosslinked polymer material has a Swelling Ratio of at least 5, at least 10, at least 20, at least 25, at least 30, at least 35, at least 40, and/or 100 or higher, as measured in Fasted State Simulated Intestinal Fluid (FASSIF).

Embodiment 4: The dosage form according to any preceding Embodiment, wherein the ionically chargeable hydrogel material comprises a plurality of ionically chargeable functional groups.

Embodiment 5: The dosage form according to any preceding Embodiment, wherein the ionically chargeable hydrogel material comprises a plurality of ionically chargeable functional groups having a net cationic charge at the intestinal pH.

Embodiment 6: The dosage form according to any one of Embodiments 1-4, wherein the ionically chargeable hydrogel material comprises a plurality of ionically chargeable functional groups having a net anionic charge at the intestinal pH.

Embodiment 7: The dosage form according to Embodiment 6, wherein the ionically chargeable hydrogel material comprises a plurality of ionically chargeable functional groups selected from the group consisting of sulfonate groups, sulfate groups, carboxylate groups, phosphate groups, and combinations thereof.

Embodiment 8: The dosage form according to any one of Embodiments 1-5, wherein the ionically chargeable hydrogel material and the ionically chargeable active agent both have a net cationic charge at the intestinal pH.

Embodiment 9: The dosage form according to any one of Embodiments 1-4, 6 and 7, wherein the ionically chargeable hydrogel material and the ionically chargeable active agent both have a net anionic charge at the intestinal pH.

Embodiment 10: The dosage form according to any preceding Embodiment, wherein the ionically chargeable active agent comprises a plurality of ionically chargeable groups.

Embodiment 11: The dosage form according to any preceding Embodiment, wherein the ionically chargeable active agent is at least one of a peptide or modified peptide, and polynucleotide, having a molecular weight of at least 500 g/mol, at least 1000 g/mol, at least 2000 g/mol, at least 4000 g/mol, at least 5000 g/mol, and/or up to 10,000 g/mol.

Embodiment 12: The dosage form according to any preceding Embodiment, wherein when the dosage form is dispersed in water, the concentration of the ionically chargeable active agent 10 minutes after dispersal in water is at least 20% higher than when the same dosage form without the crosslinked polymer is dispersed in water for the same time period.

Embodiment 13: The dosage form according to any preceding Embodiment, wherein when the dosage form is dispersed in water, the concentration of the ionically chargeable active agent 30 minutes after dispersal in water is at least 50% higher than when the same dosage form without the crosslinked polymer is dispersed in water for the same time period.

Embodiment 14: The dosage form according to any preceding Embodiment, wherein when the dosage form is dispersed in water, the concentration of the ionically chargeable active agent 60 minutes after dispersal in water is at least 100% higher than when the same dosage form without the crosslinked polymer is dispersed in water for the same time period.

Embodiment 15: The dosage form according to any preceding Embodiment, wherein when the dosage form is dispersed in water, the concentration of the ionically chargeable active agent 90 minutes after dispersal in water is at least 200% higher than when the same dosage form without the crosslinked polymer is dispersed in water for the same time period.

Embodiment 16: The dosage form according to any preceding Embodiment, wherein when the dosage form is dispersed in water, the concentration of the ionically chargeable active agent 120 minutes after dispersal in water is up to 300% higher than when the same dosage form without the crosslinked polymer is dispersed in water for the same time period.

Embodiment 17: The dosage form according to any preceding Embodiment, wherein the crosslinked polymer material swells at least 100%, at least 200%, at least 500%, at least 1000%, at least 2000%, at least 3000%, at least 4000%, at least 5000%, at least 8000%, at least 10,000% and/or at least 15,000% by weight in deionized water.

Embodiment 18: The dosage form according to any preceding Embodiment, wherein the crosslinked polymer material swells at least 100%, at least 200%, at least 500%, at least 1000%, at least 2000%, at least 3000%, at least 4000%, at least 5000%, at least 8000%, at least 10,000% and/or at least 15,000% by weight in FASSIF.

Embodiment 19: The dosage form according to any preceding Embodiment, wherein the crosslinked polymer material absorbs at least 2 mL, at least 3 mL, at least 4 mL, at least 5 mL, at least 8 mL, at least 10 mL, at least 15 mL, at least 20 mL, at least 50 mL, at least 100 mL, at least 125 mL, a least 150 mL, at least 175 mL, and/or at least 200 ml of deionized water.

Embodiment 20: The dosage form according to any preceding Embodiment, wherein the crosslinked polymer material and the ionically chargeable active agent have a net ionic charge with the same sign at pH 2.

Embodiment 21: The dosage form according to any preceding Embodiment, wherein the crosslinked polymer material and the ionically chargeable active agent have a net ionic charge with the same sign at pH 5.

Embodiment 22: The dosage form according to any preceding Embodiment, wherein the crosslinked polymer material and the ionically chargeable active agent have a net ionic charge with the same sign at pH 6.

Embodiment 23: The dosage form according to any preceding Embodiment, wherein the crosslinked polymer material and the ionically chargeable active agent have a net ionic charge with the same sign at pH 7.

Embodiment 24: The dosage form according to any preceding Embodiment, wherein a ratio of a fraction of the ionically chargeable active agent taken up into the crosslinked polymer material to a fraction of the water taken up into the crosslinked polymer, when the crosslinked polymer material is exposed to an aqueous solution containing the ionically chargeable active agent, is less than 0.1:1.

Embodiment 25: The dosage form according to any preceding Embodiment, further comprising a permeation enhancer.

Embodiment 26: The dosage form according to Embodiment 25, wherein the permeation enhancer has one or more ionically chargeable groups, and wherein the crosslinked polymer material, the ionically chargeable active agent, and the permeation enhancer all have a net ionic charge with the same sign at the intestinal pH in a range of 4 to 8.

Embodiment 27: The dosage form according to any one of Embodiments 25-26, wherein the crosslinked polymer material, the ionically chargeable active agent, and the permeation enhancer all have a net negative charge at an intestinal pH in a range of 4 to 8.

Embodiment 28: The dosage form according to any one of Embodiments 25-26, wherein the crosslinked polymer material, the ionically chargeable active agent, and the permeation enhancer all have a net negative charge in aqueous solution at pH of about 7.

Embodiment 29: The dosage form according to any one of Embodiments 25-26, wherein the crosslinked polymer material, the ionically chargeable active agent, and the permeation enhancer all have a net negative charge in aqueous solution at pH of about 5.

Embodiment 30: The dosage form according to any preceding Embodiment, comprising at least 50 mg, at least 100 mg, at least 200 mg, at least 300 mg, or at least 450 mg of the crosslinked polymer material, and less than 1 g of the crosslinked polymer material.

Embodiment 31: The dosage form according to any preceding Embodiment, wherein the crosslinked polymer material comprises at least 10%, at least 20%, at least 30%, at least 40%, at least 50%, at least 60%, at least 70%, or at least 90% of the dosage form by weight.

Embodiment 32: The dosage form according to any preceding Embodiment, wherein a ratio of the mass of the crosslinked polymer material in the dosage form to the mass of the ionically chargeable active agent is at least 2:1, at least 4:1, at least 8:1, at least 10:1, at least 20:1, at least 50:1, or at least 100:1.

Embodiment 33: The dosage form according to any preceding Embodiment, wherein the crosslinked polymer material contains an ionically chargeable moiety on at least 10%, at least 15%, at least 20%, at least 30%, at least 50%, at least 75%, at least 80%, at least 85%, at least 90%, or up to 99% of the monomers comprising the polymer.

Embodiment 34: The dosage form according to any preceding Embodiment, wherein the crosslinked polymer material contains a negatively chargeable moiety on at least 10%, at least 15%, at least 20%, at least 30%, at least 50%, at least 75%, at least 80%, at least 85%, at least 90%, or up to 99% of the monomers comprising the polymer.

Embodiment 35: The dosage form according to any one of Embodiments 1-33, wherein the crosslinked polymer material contains a positively chargeable moiety on at least 10%, at least 15%, at least 20%, at least 30%, at least 50%, at least 75%, at least 80%, at least 85%, at least 90%, or up to 99% of the monomers comprising the polymer.

Embodiment 36: The dosage form according to Embodiment 34, wherein the crosslinked polymer material contains a carboxylate moiety on at least 10%, at least 15%, at least 20%, at least 30%, at least 50%, at least 75%, at least 80%, at least 85%, at least 90%, or up to 99% of the monomers comprising the polymer.

Embodiment 37: The dosage form according to Embodiment 34, wherein the crosslinked polymer material contains a phosphate moiety on at least 10%, at least 15%, at least 20%, at least 30%, at least 50%, at least 75%, at least 80%, at least 85%, at least 90%, or up to 99% of the monomers comprising the polymer.

Embodiment 38: The dosage form according to Embodiment 34, wherein the crosslinked polymer material contains a sulfonate or sulfate moiety on at least 10%, at least 15%, at least 20%, at least 30%, at least 50%, at least 75%, at least 80%, at least 85%, at least 90%, or up to 99% of the monomers comprising the polymer.

Embodiment 39: The dosage form according to Embodiment 38, wherein a sulfur atom comprises at least 2%, at least 4%, at least 8%, at least 10%, or at least 16% of the dry weight of the crosslinked polymer material.

Embodiment 40: The dosage form according to any preceding claim, wherein the crosslinked polymer material comprises a residue of 2-acrylamido-2-methylpropane sulfonic acid or its pharmaceutically acceptable salt.

Embodiment 41: The dosage form according to Embodiment 40, wherein at least 10%, at least 15%, at least 20%, at least 30%, at least 45%, at least 50%, at least 60%, at least 75%, or at least 80% by weight of the crosslinked polymer material comprises the residue of 2-acrylamido-2-methylpropane sulfonic acid or its pharmaceutically acceptable salt.

Embodiment 42: The dosage form according to any preceding Embodiment, wherein the crosslinked polymer comprises a crosslinker comprising a residue of methylenebisacrylamide or its pharmaceutically acceptable salt.

Embodiment 43: The dosage form according to Embodiment 42, wherein from 0.2 wt % to 20 wt % of crosslinked polymer material comprises the residue of methylenebisacrylamide or its pharmaceutically acceptable salt.

Embodiment 44: The dosage form according to Embodiment 43, wherein from 1 wt % to 5 wt % of crosslinked polymer material comprises the residue of methylenebisacrylamide or its pharmaceutically acceptable salt.

Embodiment 45: The dosage form according to any preceding Embodiment, wherein the crosslinked polymer material comprises a residue of 4-vinylbenzenesulfonate or its pharmaceutically acceptable salt.

Embodiment 46: The dosage form according to Embodiment 45, wherein at least 10%, at least 15%, at least 20%, at least 30%, at least 45%, at least 50%, at least 60%, at least 75%, or at least 80% by weight of the crosslinked polymer material comprises the residue of 4-vinyl benzenesulfonate or its pharmaceutically acceptable salt.

Embodiment 47: The dosage form according to any preceding Embodiment, wherein the crosslinked polymer material comprises a residue of 4-vinylbenzenesulfonate or its pharmaceutically acceptable salt and a residue of 2-acrylamido-2-methylpropane sulfonic acid or its pharmaceutically acceptable salt in a ratio by weight that is within a range of 0.25:1 to 1:0.25, 0.5:1 to 1:0.5, or 0.75:1 to 1:0.75.

Embodiment 48: The dosage form according to any preceding Embodiment, wherein the ionically chargeable cross-linked polymer material comprises carboxylate and/or carboxylic acid groups.

Embodiment 49: The dosage form according to any preceding Embodiment, wherein the hydrogel particulates, before administration to a patient, contain less than 10% by weight of the ionically chargeable active agent within an internal dosage form volume occupied by the hydrogel particulates.

Embodiment 50: The dosage form according to any preceding Embodiment comprising a capsule form.

Embodiment 51: The dosage form according to any preceding Embodiment comprising a tablet form.

Embodiment 52: The dosage form according to any preceding Embodiment, wherein the protective coating comprises an enteric coating to release the ionically chargeable active agent in the small intestine.

Embodiment 53: The dosage form according to any preceding Embodiment, wherein the protective coating comprises a time release coating.

Embodiment 54: The dosage form according to any preceding Embodiment, comprising a capsule coated with an enteric coating.

Embodiment 55: The dosage form according to any preceding Embodiment, wherein the crosslinked polymer material comprises negatively chargeable functional groups having a pKa of less than 5 as in deionized water at a pH of about 7.

Embodiment 56: The dosage form according to any preceding Embodiment, wherein the crosslinked polymer material comprises negatively chargeable functional groups having a pKa of less than 3 as in deionized water at a pH of about 7.

Embodiment 57: The dosage form according to any preceding Embodiment, wherein at least 10%, at least 15%, at least 20%, at least 30%, at least 50%, at least 75%, at least 80%, at least 85%, at least 90%, or up to 99% of the monomers of the crosslinked polymer material comprise negatively chargeable functional groups having a pKa of less than 5 in deionized water at a pH of about 7.

Embodiment 58: The dosage form according to any preceding Embodiment, wherein at least 10%, at least 15%, at least 20%, at least 30%, at least 50%, at least 75%, at least 80%, at least 85%, at least 90%, or up to 99% of the monomers of the crosslinked polymer material comprise negatively chargeable functional groups having a pKa of less than 3 in deionized water at a pH of about 7.

Embodiment 59: A method for treating or ameliorating the effect of a condition in a subject, comprising administering to the subject an effective amount of the dosage form according to any preceding Embodiment.

Embodiment 60: The dosage form or method according to any preceding Embodiment, wherein the ionically chargeable hydrogel material reduces the activity of one or more proteases at the intestinal site.

Embodiment 61: The dosage form or method according to any preceding Embodiment, wherein the one or more proteases have a net ionic charge with a sign that is the opposite of a sign of a net ionic charge of the ionically chargeable hydrogel material at an intestinal pH in a range of from about 4 to about 8.

Embodiment 62: The dosage form or method according to any preceding Embodiment, wherein the ionically chargeable hydrogel material reduces the activity of one or more proteases by at least 15%, at least 20%, at least 25%, at least 30%, at least 45%, at least 50% overtime interval of 10 mins, 15 mins, 20 mins, 30 mins, 40 mins, 60 mins as determined by a Protease Activity Assay.

Embodiment 63: The dosage form or method according to any preceding Embodiment, wherein the delivery device comprising the ionically chargeable hydrogel material maintains or increases the concentration of the active agent at the intestinal site in the presence of proteases relative to a delivery device absent the ionically chargeable hydrogel material.

Embodiment 64: The dosage form or method according to any preceding Embodiment, wherein the delivery device comprising the ionically chargeable hydrogel material maintains or increases the concentration of the active agent in an Active Agent Concentration Assay in the presence of proteases over a time interval of at least 20 mins, at least 40 mins, or at least 60 mins.

Embodiment 65: The dosage form or method according to any preceding Embodiment, wherein the concentration of the active agent provided by the delivery device including the ionically chargeable hydrogel material, as determined by an Active Agent Concentration Assay, is higher than the concentration of the active agent provided by a delivery device without an ionically chargeable hydrogel material over a time interval of at least 20 mins, at least 40 mins, or at least 60 mins.

Embodiment 66: The dosage form or method according to Embodiment 64, wherein the proteases are α-chymotrypsin, trypsin type 1, or a combination thereof.

Embodiment 67: The dosage form or method according to any preceding Embodiment, wherein the hydrogel particulates have a mesh size in the range of 0.05 mm to 2 mm, and/or 0.5 mm to 2 mm.

Embodiment 68: The dosage form or method according to any preceding Embodiment, wherein the hydrogel particulates are capable of passing through a 63-micron sieve.

Embodiment 69: The dosage form or method according to any preceding Embodiment, wherein the dosage form comprises an active agent that is susceptible to degradation by a protease at the intestinal site.

Embodiment 70: A hydrogel comprising an ionically chargeable crosslinked polymer material comprising (i) at least 10 wt % of a residue of 4-vinylbenzenesulfonate or its pharmaceutically acceptable salt, (i) at least 10 wt % of a residue of 2-acrylamido-2-methylpropane sulfonic acid or its pharmaceutically acceptable salt, and (iii) from 0.2 wt % to 20 wt % of a crosslinker comprising a residue of methylenebisacrylamide or its pharmaceutically acceptable salt, wherein the ratio by weight of the residue of 4-vinylbenzenesulfonate or its pharmaceutically acceptable salt to the residue of 2-acrylamido-2-methylpropane sulfonic acid or its pharmaceutically acceptable salt is within a range of 0.25:1 to 1:0.25.

Embodiment 71: The hydrogel according to Embodiment 70, wherein at least 15%, at least 20%, at least 30%, at least 45%, or at least 50%, by weight of the crosslinked polymer material comprises the residue of 2-acrylamido-2-methylpropane sulfonic acid or its pharmaceutically acceptable salt.

Embodiment 72: The hydrogel according to any of Embodiments 70-71, wherein no more than 90%, 80%, 70%, 60%, 55%, 50%, 45%, 30% or 20% by weight of the crosslinked polymer material comprises the residue of 2-acrylamido-2-methylpropane sulfonic acid or its pharmaceutically acceptable salt.

Embodiment 73: The hydrogel according to any of Embodiments 70-72, wherein from 0.2 wt % to 20 wt % of crosslinked polymer material comprises the residue of methylenebisacrylamide or its pharmaceutically acceptable salt.

Embodiment 74: The hydrogel according to any of Embodiments 70-73, wherein from 1 wt % to 5 wt % of crosslinked polymer material comprises the residue of methylenebisacrylamide or its pharmaceutically acceptable salt.

Embodiment 75: The hydrogel to any of Embodiments 70-74, wherein at least 15%, at least 20%, at least 30%, at least 45%, or at least 50% by weight of the crosslinked polymer material comprises the residue of 4-vinyl benzenesulfonate or its pharmaceutically acceptable salt.

Embodiment 76: The hydrogel according to any of Embodiments 70-75, wherein no more than 90%, 80%, 70%, 60%, 55%, 50%, 45%, 30% or 20% by weight of the crosslinked polymer material comprises the residue of 4-vinyl benzenesulfonate or its pharmaceutically acceptable salt.

Embodiment 77: The hydrogel according to any of Embodiments 70-76, wherein the crosslinked polymer material comprises the residue of 4-vinylbenzenesulfonate or its pharmaceutically acceptable salt and the residue of 2-acrylamido-2-methylpropane sulfonic acid or its pharmaceutically acceptable salt in a ratio by weight that is within a range of 0.5:1 to 1:0.5, or 0.75:1 to 1:0.75.

Embodiment 78: The hydrogel according to any of Embodiments 70-77, wherein the crosslinked polymer material has a Swelling Ratio of at least 10, at least 20, at least 25, at least 30, at least 40, at least 50, at least 60, at least 75, at least 85, at least 100, at least 110, at least 120, and/or 200 or higher, as measured in deionized water.

Embodiment 79: The hydrogel according to any of Embodiments 70-78, wherein the crosslinked polymer material has a Swelling Ratio of at least 5, at least 10, at least 20, at least 25, at least 30, at least 35, at least 40, and/or 100 or higher, as measured in Fasted State Simulated Intestinal Fluid (FASSIF).

Embodiment 80: The hydrogel according to any of Embodiments 70-79, wherein the ionically chargeable hydrogel material comprises a plurality of ionically chargeable functional groups having a net anionic charge at the intestinal pH.

Embodiment 81: The hydrogel according to any of Embodiments 70-80, wherein the crosslinked polymer material swells at least 100%, at least 200%, at least 500%, at least 1000%, at least 2000%, at least 3000%, at least 4000%, at least 5000%, at least 8000%, at least 10,000% and/or at least 15,000% by weight in deionized water.

Embodiment 82: The hydrogel according to any of Embodiments 70-81, wherein the crosslinked polymer material swells at least 100%, at least 200%, at least 500%, at least 1000%, at least 2000%, at least 3000%, at least 4000%, at least 5000%, at least 8000%, at least 10,000% and/or at least 15,000% by weight in FASSIF.

Embodiment 83: The hydrogel according to any of Embodiments 70-82, wherein the crosslinked polymer material absorbs at least 2 mL, at least 3 mL, at least 4 mL, at least 5 mL, at least 8 mL, at least 10 mL, at least 15 mL, at least 20 mL, at least 50 mL, at least 100 mL, at least 125 mL, a least 150 mL, at least 175 mL, and/or at least 200 ml of deionized water.

Embodiment 84: The hydrogel according to any of Embodiments 70-83, wherein the crosslinked polymer material has a net negative ionic charge at pH 2.

Embodiment 85: The hydrogel according to any of Embodiments 70-84, wherein the crosslinked polymer material has a net negative ionic charge at pH 5.

Embodiment 86: The hydrogel according to any of Embodiments 70-85, wherein the crosslinked polymer material has a net negative ionic charge at pH 6.

Embodiment 87: The hydrogel according to any of Embodiments 70-86, wherein the crosslinked polymer material has a net anionic charge at pH 7.

Embodiment 88: The hydrogel according to any of Embodiments 70-87, wherein the crosslinked polymer material contains negatively chargeable moiety on at least 10%, at least 15%, at least 20%, at least 30%, at least 50%, at least 75%, at least 80%, at least 85%, at least 90%, or up to 99% of the monomers comprising the polymer.

Embodiment 89: The hydrogel according to any of Embodiments 70-88, wherein the crosslinked polymer material contains a sulfonate or sulfate moiety on at least 10%, at least 15%, at least 20%, at least 30%, at least 50%, at least 75%, at least 80%, at least 85%, at least 90%, or up to 99% of the monomers comprising the polymer.

Embodiment 90: The hydrogel according to any of Embodiments 70-89, wherein a sulfur atom comprises at least 2%, at least 4%, at least 8%, at least 10%, or at least 16% of the dry weight of the crosslinked polymer material.

Embodiment 91: The hydrogel according to any of Embodiments 70-90, wherein the ionically chargeable crosslinked polymer is in the form of hydrogel particulates have a mesh size in the range of 0.05 mm to 2 mm, and/or 0.5 mm to 2 mm.

Embodiment 92: The hydrogel according to any of Embodiments 70-91, wherein at least 10%, at least 15%, at least 20%, at least 30%, at least 50%, at least 75%, at least 80%, at least 85%, at least 90%, or up to 99% of the monomers of the crosslinked polymer material comprise negatively chargeable functional groups having a pKa of less than 5 in deionized water at a pH of about 7.

Embodiment 93: The hydrogel according to any of Embodiments 70-92, wherein at least 10%, at least 15%, at least 20%, at least 30%, at least 50%, at least 75%, at least 80%, at least 85%, at least 90%, or up to 99% of the monomers of the crosslinked polymer material comprise negatively chargeable functional groups having a pKa of less than 3 in deionized water at a pH of about 7.

Embodiment 94: The hydrogel according to any of Embodiments 70-93, wherein the ionically chargeable hydrogel material reduces the activity of one or more proteases by at least 15%, at least 20%, at least 25%, at least 30%, at least 45%, at least 50% over time interval of 10 mins, 15 mins, 20 mins, 30 mins, 40 mins, 60 mins as determined by a Protease Activity Assay.

Embodiment 95: The hydrogel according to any of Embodiments 70-94, wherein the ionically chargeable hydrogel material maintains or increases the concentration of an active agent in an Active Agent Concentration Assay in the presence of proteases over a time interval of at least 20 mins, at least 40 mins, or at least 60 mins.

Embodiment 96: The hydrogel according to any of Embodiments 70-95, wherein the hydrogel is in the form of hydrogel particulates that are capable of passing through a 63-micron sieve.

Embodiment 97: The dosage form according to any of Embodiments 1-69, comprising the hydrogel of any of Embodiments 70-96.

Embodiment 98: A method of treatment according to any of Embodiments 59-69, comprising administering the dosage form according to any of Embodiments 1-69 and 97, comprising the hydrogel of any of Embodiments 70-96

### INCORPORATION BY REFERENCE

All patents and patent application publications mentioned herein, are hereby incorporated by reference in their entirety for all purposes as if each individual patent and/or patent application publication was specifically and individually incorporated by reference. In case of conflict, the instant application, including any definitions herein, will control.

### EQUIVALENTS

While specific embodiments have been discussed, the above specification is illustrative and not restrictive. Many variations will become apparent to those skilled in the art upon review of this specification. The full scope of the embodiments should be determined by reference to the claims, along with their full scope of equivalents, and the specification, along with such variations.

What is claimed is:

1. A pharmaceutically acceptable oral dosage form for delivery of an active agent to an intestinal site, the dosage form comprising:
a delivery device comprising:
a plurality of hydrogel particulates comprising ionically chargeable hydrogel material, the ionically chargeable hydrogel material comprising a crosslinked polymer material having a Swelling Ratio of at least 5 in deionized water; and
an ionically chargeable active agent, the ionically chargeable active agent having a net ionic charge with a sign that is the same as a sign of a net ionic charge of the ionically chargeable hydrogel at an intestinal pH in a range of from about 4 to about 8; and
a protective coating covering the delivery device.

2. The dosage form according to claim 1, wherein the crosslinked polymer material has a Swelling Ratio of at least 10, at least 20, at least 25, at least 30, at least 40, at least 50, at least 60, at least 75, at least 85, at least 100, at least 110, at least 120, and/or 200 or higher, as measured in deionized water.

3. The dosage form according to any preceding claim, wherein the crosslinked polymer material has a Swelling Ratio of at least 5, at least 10, at least 20, at least 25, at least 30, at least 35, at least 40, and/or 100 or higher, as measured in Fasted State Simulated Intestinal Fluid (FASSIF).

4. The dosage form according to any preceding claim, wherein the ionically chargeable hydrogel material comprises a plurality of ionically chargeable functional groups.

5. The dosage form according to any preceding claim, wherein the ionically chargeable hydrogel material comprises a plurality of ionically chargeable functional groups having a net cationic charge at the intestinal pH.

6. The dosage form according to any one of claims 1-4, wherein the ionically chargeable hydrogel material comprises a plurality of ionically chargeable functional groups having a net anionic charge at the intestinal pH.

7. The dosage form according to claim 6, wherein the ionically chargeable hydrogel material comprises a plurality of ionically chargeable functional groups selected from the group consisting of sulfonate groups, sulfate groups, carboxylate groups, phosphate groups, and combinations thereof.

8. The dosage form according to any one of claims 1-5, wherein the ionically chargeable hydrogel material and the ionically chargeable active agent both have a net cationic charge at the intestinal pH.

9. The dosage form according to any one of claims 1-4, 6 and 7, wherein the ionically chargeable hydrogel material and the ionically chargeable active agent both have a net anionic charge at the intestinal pH.

10. The dosage form according to any preceding claim, wherein the ionically chargeable active agent comprises a plurality of ionically chargeable groups.

11. The dosage form according to any preceding claim, wherein the ionically chargeable active agent is at least one of a peptide or modified peptide, and polynucleotide, having a molecular weight of at least 500 g/mol, at least 1000 g/mol, at least 2000 g/mol, at least 4000 g/mol, at least 5000 g/mol, and/or up to 10,000 g/mol.

12. The dosage form according to any preceding claim, wherein when the dosage form is dispersed in water, the concentration of the ionically chargeable active agent 10 minutes after dispersal in water is at least 20% higher than when the same dosage form without the crosslinked polymer is dispersed in water for the same time period.

13. The dosage form according to any one of claim 1-11, wherein when the dosage form is dispersed in water, the concentration of the ionically chargeable active agent 30 minutes after dispersal in water is at least 50% higher than when the same dosage form without the crosslinked polymer is dispersed in water for the same time period.

14. The dosage form according to any one of claim 1-11, wherein when the dosage form is dispersed in water, the concentration of the ionically chargeable active agent 60 minutes after dispersal in water is at least 100% higher than when the same dosage form without the crosslinked polymer is dispersed in water for the same time period.

15. The dosage form according to any one of claim 1-11, wherein when the dosage form is dispersed in water, the concentration of the ionically chargeable active agent 90 minutes after dispersal in water is at least 200% higher than when the same dosage form without the crosslinked polymer is dispersed in water for the same time period.

16. The dosage form according to any one of claim 1-11, wherein when the dosage form is dispersed in water, the concentration of the ionically chargeable active agent 120 minutes after dispersal in water is up to 300% higher than when the same dosage form without the crosslinked polymer is dispersed in water for the same time period.

17. The dosage form according to any preceding claim, wherein the crosslinked polymer material swells at least 100%, at least 200%, at least 500%, at least 1000%, at least 2000%, at least 3000%, at least 4000%, at least 5000%, at least 8000%, at least 10,000% and/or at least 15,000% by weight in deionized water.

18. The dosage form according to any one of claims 1-16, wherein the crosslinked polymer material swells at least 100%, at least 200%, at least 500%, at least 1000%, at least 2000%, at least 3000%, at least 4000%, at least 5000%, at least 8000%, at least 10,000% and/or at least 15,000% by weight in FASSIF.

19. The dosage form according to any preceding claim, wherein the crosslinked polymer material absorbs at least 2 mL, at least 3 mL, at least 4 mL, at least 5 mL, at least 8 m L, at least 10 mL, at least 15 m L, at least 20 m L, at least 50 m L, at least 100 m L, at least 125 mL, a least 150 mL, at least 175 mL, and/or at least 200 ml of deionized water.

20. The dosage form according to any preceding claim, wherein the crosslinked polymer material and the ionically chargeable active agent have a net ionic charge with the same sign at pH 2.

21. The dosage form according to any one of claims 1-19, wherein the crosslinked polymer material and the ionically chargeable active agent have a net ionic charge with the same sign at pH 5.

22. The dosage form according to any one of claims 1-19, wherein the crosslinked polymer material and the ionically chargeable active agent have a net ionic charge with the same sign at pH 6.

23. The dosage form according to any one of claims 1-19, wherein the crosslinked polymer material and the ionically chargeable active agent have a net ionic charge with the same sign at pH 7.

24. The dosage form according to any preceding claim, wherein a ratio a fraction of the ionically chargeable active agent taken up into the crosslinked polymer material to a fraction of the water taken up into the crosslinked polymer, when the crosslinked polymer material is exposed to an aqueous fluid containing the ionically chargeable active agent, is less than 0.1:1.

25. The dosage form according to any preceding claim, further comprising a permeation enhancer.

26. The dosage form according to claim 25, wherein the permeation enhancer has one or more ionically chargeable groups, and wherein the crosslinked polymer material, the ionically chargeable active agent, and the permeation enhancer all have a net ionic charge with the same sign at the intestinal pH in a range of 4 to 8.

27. The dosage form according to any one of claims 25-26, wherein the crosslinked polymer material, the ionically chargeable active agent, and the permeation enhancer all have a net negative charge at an intestinal pH in a range of 4 to 8.

28. The dosage form according to any one of claims 25-26, wherein the crosslinked polymer material, the ionically chargeable active agent, and the permeation enhancer all have a net negative charge in aqueous solution at pH of about 7.

29. The dosage form according to any one of claims 25-26, wherein the crosslinked polymer material, the ionically chargeable active agent, and the permeation enhancer all have a net negative charge in aqueous solution at pH of about 5.

30. The dosage form according to any preceding claim, comprising at least 50 mg, at least 100 mg, at least 200 mg, at least 300 mg, or at least 450 mg of the crosslinked polymer material, and less than 1 g of the crosslinked polymer material.

31. The dosage form according to any preceding claim, wherein the crosslinked polymer material comprises at least 10%, at least 20%, at least 30%, at least 40%, at least 50%, at least 60%, at least 70%, or at least 90% of the dosage form by weight.

32. The dosage form according to any preceding claim, wherein a ratio of the mass of the crosslinked polymer material in the dosage form to the mass of the ionically chargeable active agent is at least 2:1, at least 4:1, at least 8:1, at least 10:1, at least 20:1, at least 50:1, or at least 100:1.

33. The dosage form according to any preceding claim, wherein the crosslinked polymer material contains an ionically chargeable moiety on at least 10%, at least 15%, at least 20%, at least 30%, at least 50%, at least 75%, at least 80%, at least 85%, at least 90%, or up to 99% of the monomers comprising the polymer.

34. The dosage form according to any preceding claim, wherein the crosslinked polymer material contains a negatively chargeable moiety on at least 10%, at least 15%, at least 20%, at least 30%, at least 50%, at least 75%, at least 80%, at least 85%, at least 90%, or up to 99% of the monomers comprising the polymer.

35. The dosage form according to any one of claims 1-33, wherein the crosslinked polymer material contains a positively chargeable moiety on at least 10%, at least 15%, at least 20%, at least 30%, at least 50%, at least 75%, at least 80%, at least 85%, at least 90%, or up to 99% of the monomers comprising the polymer.

36. The dosage form according to claim 34, wherein the crosslinked polymer material contains a carboxylate moiety on at least 10%, at least 15%, at least 20%, at least 30%, at least 50%, at least 75%, at least 80%, at least 85%, at least 90%, or up to 99% of the monomers comprising the polymer.

37. The dosage form according to claim 34, wherein the crosslinked polymer material contains a phosphate moiety on at least 10%, at least 15%, at least 20%, at least 30%, at least 50%, at least 75%, at least 80%, at least 85%, at least 90%, or up to 99% of the monomers comprising the polymer.

38. The dosage form according to claim 34, wherein the crosslinked polymer material contains a sulfonate or sulfate moiety on at least 10%, at least 15%, at least 20%, at least 30%, at least 50%, at least 75%, at least 80%, at least 85%, at least 90%, or up to 99% of the monomers comprising the polymer.

39. The dosage form according to claim 38, wherein a sulfur atom comprises at least 2%, at least 4%, at least 8%, at least 10%, or at least 16% of the dry weight of the crosslinked polymer material.

40. The dosage form according to any preceding claim, wherein the crosslinked polymer material comprises a residue of 2-acrylamido-2-methylpropane sulfonic acid or its pharmaceutically acceptable salt.

41. The dosage form according to claim 40, wherein at least 10%, at least 15%, at least 20%, at least 30%, at least 45%, at least 50%, at least 60%, at least 75%, or at least 80% by weight of the crosslinked polymer material comprises the residue of 2-acrylamido-2-methylpropane sulfonic acid or its pharmaceutically acceptable salt.

42. The dosage form according to any preceding claim, wherein the crosslinked polymer comprises a crosslinker comprising a residue of methylenebisacrylamide or its pharmaceutically acceptable salt.

43. The dosage form according to claim 42, wherein from 0.2 wt % to 20 wt % of crosslinked polymer material comprises the residue of methylenebisacrylamide or its pharmaceutically acceptable salt.

44. The dosage form according to claim 43, wherein from 1 wt % to 5 wt % of crosslinked polymer material comprises the residue of methylenebisacrylamide or its pharmaceutically acceptable salt.

45. The dosage form according to any preceding claim, wherein the crosslinked polymer material comprises a residue of 4-vinylbenzenesulfonic acid or its pharmaceutically acceptable salt.

46. The dosage form according to claim 45, wherein at least 10%, at least 15%, at least 20%, at least 30%, at least 45%, at least 50%, at least 60%, at least 75%, or at least 80% by weight of the crosslinked polymer material comprises the residue of 4-vinyl benzenesulfonic acid or its pharmaceutically acceptable salt.

47. The dosage form according to any preceding claim, wherein the crosslinked polymer material comprises a residue of 4-vinylbenzenesulfonic acid or its pharmaceutically acceptable salt and a residue of 2-acrylamido-2-methylpropane sulfonic acid or its pharmaceutically acceptable salt in a ratio by weight that is within a range of 0.25:1 to 1:0.25, 0.5:1 to 1:0.5, or 0.75:1 to 1:0.75.

48. The dosage form according to any preceding claim, wherein the ionically chargeable cross-linked polymer material comprises carboxylate and/or carboxylic acid groups.

49. The dosage form according to any preceding claim, wherein the hydrogel particulates, before administration to a patient, contain less than 10% by weight of the ionically chargeable active agent within an internal dosage form volume occupied by the hydrogel particulates.

50. The dosage form according to claim 40, wherein the hydrogel particulates have a mesh size in the range of 0.05 mm to 2 mm, and/or 0.5 mm to 2 mm.

51. The dosage form according to any preceding claim comprising a capsule form.

52. The dosage form according to any preceding claim comprising a tablet form.

53. The dosage form according to any preceding claim, wherein the protective coating comprises an enteric coating to release the ionically chargeable active agent in the small intestine.

54. The dosage form according to any preceding claim, wherein the protective coating comprises a time release coating.

55. The dosage form according to any preceding claim, comprising a capsule coated with an enteric coating.

56. The dosage form according to any preceding claim, wherein the crosslinked polymer material comprises negatively chargeable functional groups having a pKa of less than 5 as in deionized water at a pH of about 7.

57. The dosage form according to any preceding claim, wherein the crosslinked polymer material comprises negatively chargeable functional groups having a pKa of less than 3 as in deionized water at a pH of about 7.

58. The dosage form according to claim 51, wherein at least 10%, at least 15%, at least 20%, at least 30%, at least 50%, at least 75%, at least 80%, at least 85%, at least 90%, or up to 99% of the monomers of the crosslinked polymer material comprise negatively chargeable functional groups having a pKa of less than 5 in deionized water at a pH of about 7.

59. The dosage form according to claim 52, wherein at least 10%, at least 15%, at least 20%, at least 30%, at least 50%, at least 75%, at least 80%, at least 85%, at least 90%, or up to 99% of the monomers of the crosslinked polymer material comprise negatively chargeable functional groups having a pKa of less than 3 in deionized water at a pH of about 7.

60. A method for treating or ameliorating the effect of a condition in a subject, comprising administering to the subject an effective amount of the dosage form according to claim 1.

61. The dosage form or method according to any preceding claim, wherein the ionically chargeable hydrogel material reduces the activity of one or more proteases at the intestinal site.

62. The dosage form or method according to any preceding claim, wherein the one or more proteases have a net ionic charge with a sign that is the opposite of a sign of a net ionic charge of the ionically chargeable hydrogel material at an intestinal pH in a range of from about 4 to about 8.

63. The dosage form or method according to any preceding claim, wherein the ionically chargeable hydrogel material reduces the activity of one or more proteases by at least 15%, at least 20%, at least 25%, at least 30%, at least 45%, at least 50% over time interval of 10 mins, 15 mins, 20 mins, 30 mins, 40 mins, 60 mins as determined by a Protease Activity Assay.

64. The dosage form or method according to any preceding claim, wherein the delivery device comprising the ionically chargeable hydrogel material maintains or increases the concentration of the active agent at the intestinal site in the presence of proteases relative to a delivery device absent the ionically chargeable hydrogel material.

65. The dosage form or method according to claim 65, wherein the delivery device comprising the ionically chargeable hydrogel material maintains or increases the concentration of the active agent in an Active Agent Concentration Assay in the presence of proteases over a time interval of at least 20 mins, at least 40 mins, or at least 60 mins.

66. The dosage form or method according to claim 66, wherein the concentration of the active agent provided by the delivery device including the ionically chargeable hydrogel material, as determined by an Active Agent Concentration Assay, is higher than the concentration of the active agent provided by a delivery device without an ionically chargeable hydrogel material over a time interval of at least 20 mins, at least 40 mins, or at least 60 mins.

67. The dosage form or method according to claim 66, wherein the proteases are α-chymotrypsin, trypsin type 1, or a combination thereof.

68. The dosage form or method according to any preceding claim, wherein the hydrogel particulates have a mesh size in the range of 0.05 mm to 2 mm, and/or 0.5 mm to 2 mm.

69. The dosage form or method according to any preceding claim, wherein the hydrogel particulates are capable of passing through a 63-micron sieve.

70. The dosage form or method of any preceding claim, wherein the dosage form comprises an active agent that is susceptible to degradation by a protease at the intestinal site.

71. The dosage form or method of claim 70, wherein the proteases comprise α-chymotrypsin, trypsin type 1, or a combination thereof.

72. A hydrogel comprising an ionically chargeable crosslinked polymer material comprising (i) at least 10 wt % of a residue of 4-vinylbenzenesulfonic acid or its pharmaceutically acceptable salt, (i) at least 10 wt % of a residue of 2-acrylamido-2-methylpropane sulfonic acid or its pharmaceutically acceptable salt, and (iii) from 0.2 wt % to 20 wt % of a crosslinker comprising a residue of methylenebisacrylamide or its pharmaceutically acceptable salt, wherein the ratio by weight of the residue of 4-vinylbenzenesulfonic acid or its pharmaceutically acceptable salt to the residue of 2-acrylamido-2-methylpropane sulfonic acid or its pharmaceutically acceptable salt is within a range of 0.25:1 to 1:0.25.

73. The hydrogel according to claim 72, wherein at least 15%, at least 20%, at least 30%, at least 45%, or at least 50% by weight of the crosslinked polymer material comprises the residue of 2-acrylamido-2-methylpropane sulfonic acid or its pharmaceutically acceptable salt.

74. The hydrogel according to any of claims 72-73, wherein no more than 90%, 80%, 70%, 60%, 55%, 50%, 45%, 30% or 20% by weight of the crosslinked polymer material comprises the residue of 2-acrylamido-2-methylpropane sulfonic acid or its pharmaceutically acceptable salt.

75. The hydrogel according to any of claims 72-74, wherein from 0.2 wt % to 20 wt % of crosslinked polymer material comprises the residue of methylenebisacrylamide or its pharmaceutically acceptable salt.

76. The hydrogel according to any of claims 72-75, wherein from 1 wt % to 5 wt % of crosslinked polymer material comprises the residue of methylenebisacrylamide or its pharmaceutically acceptable salt.

77. The hydrogel to any of claims 72-76, wherein at least 15%, at least 20%, at least 30%, at least 45%, or at least 50% by weight of the crosslinked polymer material comprises the residue of 4-vinylbenzenesulfonic acid or its pharmaceutically acceptable salt.

78. The hydrogel to any of claims 72-77, wherein no more than 90%, 80%, 70%, 60%, 55%, 50%, 45%, 30% or 20% by weight of the crosslinked polymer material comprises the residue of 4-vinylbenzenesulfonic acid or its pharmaceutically acceptable salt.

79. The hydrogel according to any of claims 72-78, wherein the crosslinked polymer material comprises the residue of 4-vinylbenzenesulfonic acid or its pharmaceutically acceptable salt and the residue of 2-acrylamido-2-methylpropane sulfonic acid or its pharmaceutically acceptable salt in a ratio by weight that is within a range of 0.5:1 to 1:0.5, or 0.75:1 to 1:0.75.

80. The dosage form according to any of claims 1-71, comprising the hydrogel of any of claims 72-79.

81. A method of treatment according to any of claims 60-71, comprising administering the dosage from according to any of claims 1-71 comprising the hydrogel of any of claims 72-79.
